(19) United States
(12) Patent Application Publication
(10) Pub. No.: US 2009/0016989 A1
(43) Pub. Date: Jan. 15, 2009

(54) ANTIBODIES TO HUMAN SOMATOSTATIN RECEPTOR AND METHODS OF USE

(76) Inventor: Frank Leu, Metuchen, NJ (US)

Correspondence Address: DARBY & DARBY P.C., P.O. BOX 770, Church Street Station, New York, NY, 10008-0770, US

(21) Appl. No.: 12/109,742

(22) Filed: Apr. 25, 2008

Related U.S. Application Data
(60) Provisional application No. 60/913,970, filed on Apr. 25, 2007.
(60) Provisional application No. 60/951,085, filed on Jul. 20, 2007.

(51) Int. Cl. A61K 38/21; C07K 16/28; C12N 9/00; A61P 35/00; A61K 39/00; C12N 5/06; G01N 33/566; A61K 39/395; A61K 39/44

(52) U.S. Cl. 424/85.4; 530/387.9; 530/391.3; 435/183; 530/391.7; 424/185.1; 435/375; 436/501; 424/139.1; 424/178.1

(57) ABSTRACT

The invention provides isolated antibodies, including monoclonal and polyclonal antibodies, and the antigen-binding portions thereof, that specifically bind to the extracellular loop 2 (ecl2) of human somatostatin receptor (hSSTR) subtype 1, 2, 3, 4, or 5. The antibodies and antigen-binding portions of the present invention can possess hSSTR agonist-like and/or antagonist-like properties. The invention further provides methods of making the antibodies and antigen-binding portions thereof, and methods of using the antibodies and antigen-binding portions for diagnosing and treating various indications, including cancer and carcinoid syndrome.

This application claims priority under 35 U.S.C. §119(e) to U.S. Provisional Patent Application Ser. No. 60/913,970, filed Apr. 25, 2007 and to U.S. Provisional Patent Application Ser. No. 60/951,085, filed Jul. 20, 2007. These applications are incorporated herein by reference in their entireties.

### FIELD OF THE INVENTION

The field of the invention relates generally to isolated antibodies and antigen-binding portions thereof that specifically bind to the extracellular loop 2 (ecl2) of human somatostatin receptor (hSSTR) subtype 1, 2, 3, 4, or 5. The antibodies and antigen-binding portions of the present invention can possess SSTR agonist-like and/or antagonist-like properties. The field of the invention further relates to methods of making the antibodies and antigen-binding portions thereof, and methods of using the antibodies and antigen-binding portions for diagnosing and treating various indications, including cancer and carcinoid syndrome.

### BACKGROUND

The human somatostatin receptor (hSSTR) belongs to a class of seven-transmembrane G protein-coupled receptors (GPCR). hSSTR has five subtypes: hSSTR subtype 1, hSSTR subtype 2, hSSTR subtype 3, hSSTR subtype 4 and hSSTR subtype 5. Neuroendocrine tumors overexpress hSSTR subtype 2 and hSSTR subtype 5. (H. Lahlou, J. Guillermet, M. Hortala et al., Ann N Y Acad Sci 1014, 121 (2004); C. Susini and L. Buscail, Ann Oncol (2006)). All hSSTR subtypes are intronless, except for subtype 2. In mice, SSTR subtype 2 has a cryptic intron at the C-terminus resulting in “A” or “B” variants upon alternative splicing (T. Reisine, H. Kong, K. Raynor et al., Mol Pharmacol 44 (5), 1016 (1993)). hSSTR membrane receptors are modulated by the brain-gut peptide somatostatin (SST), and their activation negatively regulates neuroendocrine cell proliferation and hormone secretion (N. Benali, G. Ferjoux, E. Puente et al., Digestion 62 Suppl 1, 27 (2000)). SST has an in vitro half-life of only a few minutes. A longer acting SST analogue (octreotide) was developed, and is commonly used to treat neuroendocrine tumors (W. Bauer, U. Briner, W. Doepfner et al., Life Sci 31 (11), 1133 (1982)). Octreotide therapy can keep tumors static for years, but it does not remove the threat of metastasis. Consequently, surgery is often necessary to prevent or treat metastasis.

A radiolabeled form of octreotide has been used in OctreoScan® scintillography as an effective tool to image and diagnose early and metastatic neuroendocrine tumors. An elevated level of expression of hSSTR subtype 2 and/or hSSTR subtype 5 relative to normal tissues allows for effective imaging of neuroendocrine tumors, and also provides a potential means to predict the effectiveness of octreotide therapy (J. C. Reubi, J. C. Schar, B. Waser et al., Eur J Nucl Med 27 (3), 273 (2000)). Although OctreoScan® scintillography has been shown to be effective for detecting neuroendocrine tumors, it does not provide detailed information about the expression levels of individual hSSTR subtypes on neuroendocrine tumors.

It has been demonstrated that hSSTRs can form homo-dimers, hetero-dimers and hetero-oligomers (M. Rocheville et al., Science 288 (5463), 154 (2000); R. C. Patel et al., Methods 27 (4), 340 (2002); and M. Pfeiffer et al., J Biol Chem 276 (17), 14027 (2001)). Determining receptor subtype distribution using subtype-specific methods may provide valuable information leading to improved therapeutic outcomes. For example, there have been reports linking the activation of SSTR subtype 3 with the activation of p53 dependent apoptosis (K. Sharma et al.,, Mol Endocrinol 10 (12), 1688 (1996)). SSTR subtype 2 and SSTR subtype 3 are known to form hetero-dimers, and it has been postulated that this dimerization prevents the direct activation of SSTR subtype 3 by its ligand. Thus, this dimerization may retard SSTR subtype 3-mediated apoptosis (M. Pfeiffer, T. Koch, H. Schroder et al., J Biol Chem 276 (17), 14027 (2001)).

Anti-SSTR antibodies are commercially available. Most commercially available anti-SSTR antibodies are prepared against the intracellular C-terminal region of SSTR. Anti-SSTR subtype 2 polyclonal rabbit antibodies, including antibodies that bind to the extracellular region of SSTR, are available from both Chemicon International, Inc. (Temecula, Calif.), and Novus Biologicals (Littleton, Colo.). Chemicon uses amino acid residues 339-359 of rat SSTR subtype 2 (Genbank Accession number: P30680) as the antigen for preparation of its polyclonal antibodies. Novus Biologicals used the protein identified by Genbank Accession number: NM_{—}001050 as the antigen for preparation of its polyclonal antibodies. The commercially available anti-SSTR subtype 2-ecl2 antibodies appear to have been used for research only, i.e., as immunohistochemistry (IHC) reagents (See www.novusbio.com and www.chemicon.com). Commercially available anti-SSTR antibodies are generally inferior in quality because commercially available SSTR antibodies usually are derived from larger peptides (with a higher number of amino acid residues) which may result in multiple species of polyclonal antibodies yielding greater false positives when used for diagnosis or laboratory experimental procedures. See Korner et al 2005 (American Journal of Surgical Pathology) from Reubi's laboratory (M Gugger, B Waser, A Kappeler et al., Gut 53 (10), 1431 (2004)). There have been no published reports describing effective, therapeutic anti-hSSTR antibodies.

There is a need for anti-hSSTR antibodies that can provide improved diagnostic efficacy and also therapeutic efficacy.

### SUMMARY OF THE INVENTION

The present invention provides isolated monoclonal or polyclonal antibodies and antigen-binding portions thereof that specifically bind to the extracellular portion of a hSSTR. More particularly, the present invention provides monoclonal or polyclonal antibodies and antigen-binding portions thereof that bind to extracellular loop 2 (ecl2) of a hSSTR subtype.

In one embodiment, the antibodies and antigen-binding portions specifically bind to ecl2 of hSSTR subtype 2. In another embodiment, the antibodies and antigen-binding portions specifically bind to ecl2 of hSSTR subtype 5. In a further embodiment, the antibodies and antigen-binding portions specifically bind to amino acid residues 1-14 of SEQ ID NO: 1 (QWGRSSCTINWPGE) as present in ecl2 of hSSTR subtype 2. In another embodiment, the antibodies and antigen-binding portions specifically bind to amino acid residues 1-14 of SEQ ID NO: 2 (DVQEGGTCNASWPE) as present in ecl2 of hSSTR subtype 5. In an aspect of the present invention, the antibodies and antigen-binding portions thereof specifically bind to a particular subtype of hSSTR compared to other subtypes of hSSTR. For example, an anti-hSSTR subtype 2 antibody, or antigen-binding portion thereof binds to the ecl2 of hSSTR subtype 2, but not to the ecl2 of hSSTR subtypes 1, 3, 4, or 5; and an anti-hSSTR subtype 5 antibody, or antigen-binding portion thereof specifically binds to the ecl2 of hSSTR subtype 5, but not to the ecl2 of hSSTR subtypes 1, 2, 3, or 4.

The present invention further provides methods for producing monoclonal or polyclonal antibodies and antigen-binding portions thereof that specifically bind to ecl2 of hSSTR subtype 2, comprising immunizing a mammal with a polypeptide comprising amino acid residues 1-14 of SEQ ID NO: 1, and isolating an antibody or antigen-binding portion thereof that specifically binds to amino acid residues 1-14 of SEQ ID NO:1 as present in ecl2 of hSSTR subtype 2.

The present invention further provides methods for producing monoclonal or polyclonal antibodies and antigen-binding portions thereof that specifically bind to ecl2 of hSSTR subtype 5, comprising immunizing a mammal with a polypeptide comprising amino acid residues 1-14 of SEQ ID NO:2, and isolating an antibody or antigen-binding portion thereof that specifically binds to amino acid residues 1-14 of SEQ ID NO:2 as present in ecl2 of hSSTR subtype 5.

Antibodies and antigen-binding portions that specifically bind to ecl2 of hSSTR subtype 2, as provided by the present invention, may have agonist-like and/or antagonist-like properties on hSSTR subtype 2. Antibodies and antigen-binding portions that specifically bind to ecl2 of hSSTR subtype 5, as provided by the present invention, may have agonist-like and/or antagonist-like properties on hSSTR subtype 5.

Suppression of neuroendocrine dense core granule release may be of therapeutic benefit in hSSTR-related diseases and disorders. Neuroendocrine dense core granule content includes peptide hormones, biogenic amines such as but not limited to serotonin, carrier proteins, adenylate 5′-triphosphate, Ca⁺, Chromogranin A and its derived peptides. According to the present invention, neuroendocrine dense core granule content release from a cell (e.g., a cell expressing hSSTRs) can be suppressed by contacting the cell with an anti-hSSTR antibody or antigen-binding portion thereof. The present invention thus provides a method of suppressing dense core granule content release in a cell comprising contacting the cell with an anti-hSSTR subtype 2 antibody or antigen-binding portion of the present invention. The present invention further provides a method of suppressing dense core granule content release in a cell comprising contacting the cell with an anti-hSSTR subtype 5 antibody or antigen-binding portion of the present invention. According to the present invention, serotonin release from a cell (e.g., a cell expressing hSSTRs) can be suppressed by contacting the cell with an anti-hSSTR antibody or antigen-binding portion of the present invention. Suppression of inappropriate serotonin release can have the beneficial effect(s) of preventing the dilation of blood vessels, diarrhea and wheezing, abdominal pain, flushing, palpitations and low blood pressure.

The present invention thus provides a method of suppressing serotonin release in a cell comprising contacting the cell with an anti-hSSTR subtype 2 antibody or antigen-binding portion of the present invention. The present invention further provides a method of suppressing serotonin release in a cell comprising contacting the cell with an anti-hSSTR subtype 5 antibody or antigen-binding portion of the present invention.

Also according to the present invention, cell cycle arrest can be induced by contacting a cell (e.g., a cell expressing hSSTR subtype 1, 2, 3, 4, or 5) with an anti-hSSTR antibody or antigen-binding portion thereof. Cell cycle arrest is the process by which progression through the cell cycle is halted during one of the normal phases G1, S, G2, or M. The present invention thus provides a method of inducing cell cycle arrest by contacting a cell with an anti-hSSTR subtype 2 antibody or antigen-binding portion thereof. The present invention further provides a method of inducing cell cycle arrest by contacting a cell with an anti-hSSTR subtype 5 antibody or antigen-binding portion thereof.

The present invention further provides anti-hSSTR antibodies and antigen-binding portions thereof that are detectably labeled. In one embodiment, the present invention provides anti-hSSTR subtype 2 antibodies, or antigen-binding portions thereof, that are detectably labeled. In another embodiment, the present invention provides anti-hSSTR subtype 5 antibodies, or antigen-binding portions thereof, that are detectably labeled. Such detectably labeled antibodies and antigen-binding portions are particularly useful in diagnostic methods to detect the presence of hSSTR subtype 2-expressing cancer cells or hSSTR subtype 5-expressing cancer cells in a patient or in a biological sample collected from a patient.

The present invention further provides anti-hSSTR antibodies and antigen-binding portions thereof that are conjugated to a chemotherapeutic agent. Such conjugated antibodies or antigen-binding portions may have enhanced therapeutic efficacy in treating, ameliorating or preventing neuroendocrine tumors or carcinoid syndrome, or one or more symptoms thereof.

The present invention further provides pharmaceutical compositions comprising therapeutically effective amounts of an antibody or antigen-binding portion of the present invention admixed with a pharmaceutically acceptable carrier. Such pharmaceutical compositions may further comprise a second therapeutic agent, such as a chemotherapeutic agent useful for treating a proliferative disease.

The present invention further provides a method for treating a neuroendocrine tumor in a patient, comprising administering a therapeutically effective amount of an anti-hSSTR subtype 2 antibody or antigen-binding portion of the present invention to the patient. The present invention further provides a method for treating a neuroendocrine tumor in a patient, comprising administering a therapeutically effective amount of an anti-hSSTR subtype 5 antibody or antigen-binding portion of the present invention to the patient.

The present invention further provides a method for treating a carcinoid syndrome in a patient, comprising administering a therapeutically effective amount of an anti-hSSTR subtype 2 antibody or antigen-binding portion of the present invention to the patient. The present invention further provides a method for treating a carcinoid syndrome in a patient, comprising administering a therapeutically effective amount of an anti-hSSTR subtype 5 antibody or antigen-binding portion of the present invention to the patient.

The present invention further provides a method of diagnosing a neuroendocrine tumor in a patient by administering a detectably labeled anti-hSSTR subtype 2 antibody or antigen-binding portion of the present invention to a patient suspected of having hSSTR subtype 2-expressing cancer cells, and detecting the presence or localization of the labeled antibody or antigen-binding portion in situ in the tissues of the patient, or in a biological sample collected from the patient, thereby indicating the presence of hSSTR subtype 2-expressing cancer cells.

The present invention further provides a method of diagnosing a neuroendocrine tumor in a patient by administering a detectably labeled anti-hSSTR subtype 5 antibody or antigen-binding portion of the present invention to a patient suspected of having hSSTR subtype 5-expressing cancer cells, and detecting the presence or localization of the labeled antibody or antigen-binding portion in situ in the tissues of the patient, or in a biological sample collected from the patient, thereby indicating the presence of hSSTR subtype 5-expressing cancer cells.

### BRIEF DESCRIPTION OF THE DRAWINGS

FIG. 1 schematically represents a SSTR, which is a 7-transmembrane G-protein-coupled receptor.

FIG. 2 presents histograms of flow cytometry data, demonstrating that polyclonal antibodies of the present invention recognize the presence of SSTR subtype 2, SSTR subtype 3, and SSTR subtype 5 on the extracellular membrane surface of BON cells.

FIG. 3 presents a bar graph demonstrating suppression of cell growth in BON cells by anti-hSSTR subtype 2 antibodies or anti-hSSTR subtype 5 antibodies.

FIG. 4 presents bar graphs demonstrating that SSTR-ecl2 subtype 2 peptide antigen blocks the ability of anti-hSSTR subtype 2 antibodies to retard cell growth in BON cells (FIG. 4A), and that SSTR-ecl2 subtype 5 peptide antigen blocks the ability of anti-hSSTR subtype 5 (FIG. 4B) antibodies to retard cell growth in BON cells.

FIG. 5 presents a graph demonstrating that BON cells stably transfected with either shRNA hSSTR subtype 2 or shRNA hSSTR subtype 5 maintain their sensitivity to octreotide.

FIG. 6 presents bar graphs demonstrating subtype-specific inhibition of BON cell proliferation. FIG. 6A. Proliferation of sh-hSSTR subtype 2-expressing BON cells is inhibited by anti-hSSTR subtype 2 antibody. FIG. 6B. Proliferation of sh-hSSTR subtype 5-expressing BON cells is inhibited by anti-hSSTR subtype 5 antibody.

FIG. 7 presents a bar graph demonstrating an additive effect on cell growth suppression in BON cells treated with both anti-hSSTR subtype 2 and anti-hSSTR subtype 3 antibodies compared to treatment of BON cells with either antibody alone.

FIG. 8 presents a bar graph demonstrating that SSTR subtype 3-ecl2 peptide blocks the effect of anti-hSSTR subtype 3 antibody-mediated cell growth suppression in BON cells.

FIG. 9 presents histograms demonstrating increased cell death in BON cells after treatment with anti-hSSTR subtype 2, anti-hSSTR subtype 3, or anti-hSSTR subtype 5 antibodies compared to control samples treated with rabbit IgG.

FIG. 10 presents graphs demonstrating increased levels of apoptosis in BON cells treated with anti-hSSTR subtype 2 or anti-hSSTR subtype 5 antibodies after 24 hours (FIG. 10B), 48 hours (FIG. 10C), and 72 hours (FIG. 10D) compared to treatment of BON cells with rabbit IgG (negative control) or 100 nM octreotide. FIG. 10A shows BON cells treated with staurosporin after 6 hours (positive control). Quantification of the results presented in FIGS. 1A-10D is provided in Tables 1A-1D, respectively (Example 5).

FIG. 11 presents bar graphs demonstrating activation of caspase 8 (FIG. 11B) and caspase 9 (FIG. 11C), but not caspase 3 or caspase 7 (FIG. 11A), in BON cells after treatment with anti-hSSTR subtype 2 antibody, anti-hSSTR subtype 3 antibody, or anti-hSSTR subtype 5 antibody. Standard deviations are plotted as error bars in the graph. The sample size for each experiment is greater than 3.

FIG. 12 present graphs demonstrating that anti-SSTR subtype 2 antibodies alone and anti-hSSTR subtype 5 antibodies alone inhibit BON cells from entering the S phase of the cell cycle compared to cells treated with vehicle or rabbit IgG. This is similar to inhibition by octreotide on the ability of BON cells to enter the S phase.

FIG. 13 presents a bar graph demonstrating that adenylyl cyclase activity is not inhibited by anti-hSSTR subtype 2 or anti-hSSTR subtype 5 antibodies. Quantification of the results presented in FIG. 13 is provided in Table 2 (Example 5).

FIG. 14 presents graphs demonstrating cell growth suppression by anti-hSSTR subtype 2 antibodies in the presence of endogenous SST in mouse fibroblast 3T3 cells harboring the SSTR recombinant gene (FIG. 14B) compared to control cells (FIG. 14A).

FIG. 15 presents a bar graph demonstrating growth inhibition of pancreatic islet cell carcinoma cell line QGP by anti-hSSTR subtype 2 antibodies or anti-hSSTR subtype 5 antibodies.

FIG. 16 presents a bar graph demonstrating the inhibition of serotonin release by BON cells exposed to anti-hSSTR subtype 2, 3, and 5 antibodies.

### DETAILED DESCRIPTION OF THE INVENTION

### Definitions and General Techniques

Unless otherwise defined herein, scientific and technical terms used in connection with the present invention shall have the meanings that are commonly understood by those of ordinary skill in the art. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention can be generally performed according to conventional methods well known in the art and as described in various general and more specific references that are cited and discussed throughout the present specification unless otherwise indicated. See, e.g., Sambrook et al. Molecular Cloning: A Laboratory Manual. 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Standard techniques are used for chemical syntheses, chemical analyses, pharmaceutical preparation, formulation, and delivery, and treatment of patients.

As used herein, an “antibody” refers to a complete antibody (which may also include antigen-binding portions thereof) that competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions of antibodies may be produced by recombinant DNA techniques or by enzymatic or chemical cleavage of intact antibodies. Antigen-binding portions include, e.g., Fab, Fab′, F(ab′)₂, Fd, Fv, dAb, and complementarity determining region (CDR) fragments, single-chain antibodies (scFv), chimeric antibodies, diabodies and polypeptides that contain at least a portion of an antibody, which portion is sufficient to confer specific antigen binding to the polypeptide.

An intact antibody comprises at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, CH1, CH2 and CH3. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The assignment of amino acids to each of the light and heavy chain variable domains is in accordance with the definitions of Kabat, Sequences of Proteins of Immunological Interest (National Institutes of Health, Bethesda, Md. (1987 and 1991)); or Chothia & Lesk, J. Mol. Biol. 196:901-917 (1987); Chothia et al., Nature 342:878-883 (1989). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “polyclonal antibodies,” and “polyclonal antibody composition” as used herein, refer to a preparation comprising a plurality of antibodies derived from multiple B-cells that recognize different epitopes which are all directed to the same antigen. Such a preparation typically includes antibodies binding to different epitopes on the same antigen. Methods of preparing polyclonal antibodies are known in the art. Such methods typically include immunizing a mammal with an antigen, and subsequently collecting antibodies from the serum of the mammal.

The terms “monoclonal antibody” and “monoclonal antibody composition” as used herein, refer to a preparation consisting of a single species of antibody molecules with a single binding specificity and affinity for a particular epitope within the antigen. A monoclonal antibody refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof, mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

The term “human monoclonal antibody” or “humanized antibodies” refers to monoclonal antibodies that have variable and constant regions (if present) derived from human germline immunoglobulin sequences. “Humanized” antibodies refer to molecules having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, or six) which are altered with respect to the original antibody. Human monoclonal antibodies can be produced by standard methodologies, including though the preparation of an appropriate hybridoma.

The term “chimeric antibody” as used herein means an antibody that comprises regions from two or more different antibodies. In one embodiment, one or more of the CDRs are derived from an anti-hSSTR antibody. In another embodiment, all of the CDRs are derived from an anti-hSSTR antibody. In another embodiment, the CDRs from more than one human anti-hSSTR antibody are combined into a chimeric antibody. For instance, a chimeric antibody may comprise CDR1 from the light chain of a first anti-hSSTR antibody, CDR2 from the light chain of a second anti-hSSTR antibody, and CDR3 from the light chain of a third anti-hSSTR antibody. The CDRs from the heavy chain may be derived from one or more other anti-hSSTR antibodies. The framework regions may be derived from one of the same anti-hSSTR antibodies or from one or more different humans.

The term “agonist-like” as used herein means that upon the binding of an anti-hSSTR antibody or antigen-binding portion of the present invention to a subtype of SSTR, at least one biological effect typically observed when SST binds to a SSTR in vivo is induced. Such an effect can be, for example, inhibition of cellular growth or blocking of serotonin release from a cell.

The term “antagonist-like” as used herein means that upon the binding of an anti-hSSTR antibody or antigen-binding portion of the present invention to a subtype of SSTR, at least one biological effect typically elicited by an agonist of SSTR (e.g., SST), is inhibited (i.e., prevented or lessened). For example, in one embodiment, cAMP induction induced by forskolin is only moderately increased in the presence of anti-hSSTR subtype 2 and anti-hSSTR subtype 5 antibodies. This effect is different than the result that would be produced with an SST agonist, which decreases cAMP levels significantly.

Anti-hSSTR antibodies or antigen-binding portions of the present invention can have both agonist-like and antagonist-like properties on hSSTR. Such an antibody or antigen-binding portion would, upon binding to its antigen, exhibit at least one agonist-like effect and at least one-antagonist-like effect. In another embodiment, the antibody or antigen-binding portion of the present invention would exhibit therapeutic effectiveness like an agonist of hSSTR (i.e., inhibiting both cell growth and serotonin release) and also have the ability to reduce undesirable effects of agonists of hSSTR resulting from setting off cascades of second messenger responses (i.e., cAMP increase.).

The term “epitope” includes any molecular (e.g., protein) determinant capable of being specifically bound by an immunoglobulin molecule or T-cell receptor. Epitopic determinants usually consist of chemically active surface groupings of molecules, such as amino acids or sugar side chains, and usually have specific three dimensional structural characteristics, as well as specific charge characteristics.

As used herein, the terms “detectable label” and “detectably labeled” refer to a molecule or moiety that can be incorporated (e.g., by covalent bonding) into, or conjugated to, the antibody or antigen-binding portion, where the presence of the molecule or moiety (and thus the antibody or antigen-binding portion) can be detected in vivo or in a biological sample collected from the patient, by use of a standardized detection technique. For example, the detectable marker can be a radiolabeled amino acid, or a polypeptide of biotin moieties that can be detected by marked avidin, or a fluorescent marker or colorimetric enzyme that can be detected by optical or colorimetric methods. Various methods of labeling polypeptides and glycoproteins are known in the art and can be used according to the present invention. Examples of detectable labels also include, radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, and other labels such as quantum dots, nano/micro spheres, gold among others.

Antibodies or antigen-binding portions of the present invention can be conjugated to chemotherapeutic agents such as, e.g., appropriate radiolabels, or to pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof, and quantum dots, nano/micro spheres, metal particles (i.e., gold or iron) particles and carbon nanotubes among others.

As used herein, the term “subject” or “patient” primarily includes humans, but also can refer to non-human mammals including non-human primates and companion animals, among other mammals.

As used herein, the terms “specific binding” and “specifically bind” mean that an antibody or antigen-binding portion thereof of the invention preferentially binds to a particular subtype of hSSTR compared to other subtypes of hSSTR.

As used herein, the terms “isolated” and “isolating” mean that the referenced material is removed from its native environment, e.g., a cell. Thus, an isolated biological material can be free of some or all cellular components, i.e., components of the cells in which the native material naturally occurs (e.g., a cytoplasmic or membrane component).

As used herein, the phrase “as present in ecl2 of hSSTR subtype . . . ” refers to the natural configuration of the peptide having the particular sequence (e.g., amino acid residues 1-14 of SEQ ID NO:1) as it occurs in the intact and functional hSSTR protein when present in a mammalian cellular membrane under physiological conditions in cell culture or in vivo. The phrase “as present in ecl2 of hSSTR subtype . . . ” also refers to denatured conformations of hSSTR protein. Thus, for example, a polyclonal or monoclonal anti-hSSTR antibody of the present invention can bind to intact and functional hSSTR subtype 2 on a live cell and to denatured hSSTR subtype 2.

hSSTR Antibodies

Ecl2 is the largest of the three extracellular loops in hSSTR, and is the loop that is distant, and possibly furthest, from the potential N-terminal glycosylation site, which might otherwise interfere with antibody binding to hSSTR. In hSSTR subtype 5, ecl2 appears to be an important loop in SST binding (Greenwood et al., Mol. Pharmacol., 52:807-14 (1997)).

According to the present invention, ecl2 was selected for preparing anti-hSSTR polyclonal antibodies that: (i) recognize and bind to the extracellular portion of hSSTR exposed on the plasma membrane surface of live cells; (ii) possess agonist-like and/or antagonist-like properties upon binding with specific hSSTR subtypes (useful for studying SSTR signal transduction components and pathways); (iii) possess diagnostic and/or therapeutic properties; and/or (iv) serve as a SSTR subtype-specific delivery agent when coupled, e.g., with a chemotherapeutic agent.

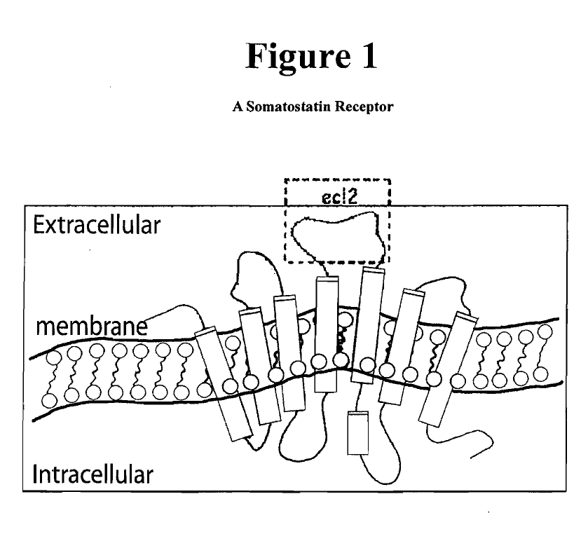

A specific portion of extracellular loop number 2 (ecl2) (FIG. 1) was used as the antigen peptide for production of antibodies according to the present invention. The ecl2 of hSSTR consists of amino acids. More specifically, a 14 amino acid peptide sequence derived from the full-length ecl2 of each hSSTR subtype was used to generate hSSTR subtype specific antibodies. The antigens of the ecl2 of each hSSTR subtype used to make subtype specific antibodies of the invention and the full length amino acid sequence of ecl2 of each hSSTR subtype are listed below:

hSSTR subtype 1:

(SEQ ID NO: 3)

TVACNMLMPEPAQR
[14 aa′s];
full length hSSTR subtype 1 ecl2:

(SEQ ID NO: 8)

SRTAANSDGTVACNMLMPEPAQRWL
[25 amino acids];
hSSTR subtype 2:

(SEQ ID NO: 1)

QWGRSSCTINWPGE
[14 aa′s];
full length hSSTR subtype 2 ecl2:

(SEQ ID NO: 6)

AGLRSNQWGRSSCTINWPGESGAWYTGF
[28 amino acids];
hSSTR subtype 3:

(SEQ ID NO: 4)

PRGMSTCHMQWPEP
[14 aa′s];
full length hSSTR subtype 3 ecl2:

(SEQ ID NO: 9)

FSGVPRGMSTCHMQWPEPAAAWRAGF
[26 amino acids];
hSSTR subtype 4:

(SEQ ID NO: 5)

DTRPARGGQAVACN
[14 aa′s];
full length hSSTR subtype 4 ecl2:

(SEQ ID NO: 10)

ADTRPARGGQAVACNLQWPHPAWSA
[25 amino acids];
hSSTR subtype 5:

(SEQ ID NO: 2)

DVQEGGTCNASWPE
[14 aa′s];
and
full length hSSTR subtype 5 ecl2:

(SEQ ID NO: 7)

ADVQEGGTCNASWPEPVGLWG
[21 amino acids].

The present invention thus provides isolated monoclonal or polyclonal antibodies and antigen-binding portions thereof that specifically bind to hSSTR subtype 1, or to hSSTR subtype 2, or to hSSTR subtype 3, or to hSSTR subtype 4 or to hSSTR subtype 5. In one embodiment, the anti-hSSTR antibodies and antigen-binding portions specifically bind to the ecl2 of hSSTR subtype 1. In another embodiment, the anti-hSSTR antibodies and antigen-binding portions specifically bind to the ecl2 of hSSTR subtype 2. In another embodiment, the anti-hSSTR antibodies and antigen-binding portions specifically bind to the ecl2 of hSSTR subtype 3. In another embodiment, the anti-hSSTR antibodies and antigen-binding portions specifically bind to the ecl2 of hSSTR subtype 4. In another embodiment, the anti-hSSTR antibodies and antigen-binding portions specifically bind to the ecl2 of hSSTR subtype 5.

In another embodiment, the anti-hSSTR antibodies and antigen-binding portions specifically bind to the ecl2 of hSSTR subtype 2, but do not bind to the ecl2 of hSSTR subtype 1, 3, 4, or 5. In another embodiment, the anti-hSSTR antibodies and antigen-binding portions specifically bind to the ecl2 of hSSTR subtype 3, but do not bind to the ecl2 of hSSTR subtype 1, 2, 4, or 5. In another embodiment, the anti-hSSTR antibodies and antigen-binding portions specifically bind to the ecl2 of hSSTR subtype 4, but do not bind to the ecl2 of hSSTR subtype 1, 2, 3, or 5. In another embodiment, the anti-hSSTR antibodies and antigen-binding portions specifically bind to the ecl2 of hSSTR subtype 5, but do not bind to the ecl2 of hSSTR subtype 1, 2, 3, or 4.

In a particular embodiment, the anti-hSSTR antibodies and antigen-binding portions thereof specifically bind to amino acid residues 1-14 of SEQ ID NO: 1 as present in ecl2 of hSSTR subtype 2. In another particular embodiment, the anti-hSSTR antibodies and antigen-binding portions thereof specifically bind to amino acid residues 1-14 of SEQ ID NO: 2 as present in ecl2 of hSSTR subtype 5.

Anti-hSSTR antibodies and antigen-binding portions thereof according to the present invention can inhibit the growth of cancer cells in vitro or in vivo. In one embodiment, the inhibition of tumor growth is detectable 14 days after initial treatment of a patient with a therapeutically effective amount of an anti-hSSTR antibody or antigen-binding portion of the present invention. In another embodiment, the inhibition of cancer growth is detectable 7 days after initial treatment of a patient with a therapeutically effective amount of an anti-hSSTR antibody or antigen-binding portion of the present invention.

In certain embodiments, a second therapeutic agent is co-administered to the patient with the hSSTR antibody or antigen-binding portion. The second therapeutic agent can be any therapeutic agent for treating cancer such as a chemotherapeutic agent(e.g., fluorouracil (5-FU), doxorubicin, streptozotocin, dacarbazine, cis-platin, a taxane such as taxol, or a biologic agent such as an interferon) or radiotherapy (e.g., external beam radiation therapy, internal radiation therapy (brachytherapy), radioimmunotherapy, intra-operative radiotherapy, interstitial radiotherapy, endocavitary radiotherapy).

Upon contact of an anti-hSSTR antibody or antigen binding portion of the present invention with a biological sample expressing the particular hSSTR subtype to which the antibody is directed, at least one of the following effects on a cell or a tissue of the biological sample can subsequently be observed compared to the same type of cell or a tissue expressing the same subtype of hSSTR that has not been contacted with the antibody or the antigen-binding portion thereof: (1) suppression of serotonin release; (2) induction of activation of caspase 8; (3) induction of activation of caspase 9; (4) suppression of cell growth; (5) induction of or increase in cell death; (6) induction of or increase in programmed cell death by apoptosis; (7) induction of cell cycle arrest of a cell or cells by maintaining the cell or cells in the G0/G1 phase; (8) induction of cell cycle arrest of a cell or cells by maintaining the cell or cells in the G2/M phase; (9) induction of cell cycle arrest of a cell or cells by preventing the cell or cells from entering the S phase; or (10) suppression of neuroendocrine dense core granule release. Cell death can occur in a biological sample expressing an hSSTR that has been contacted with the antibody or the antigen-binding portion thereof by apoptosis, necrosis, or autophagic cell death. In one embodiment, the anti-hSSTR antibodies have at least two of effects (1)-(10). In another embodiment, the anti-hSSTR antibodies have at least three of effects (1)-(10).

In one embodiment, the anti-hSSTR antibody or antigen-binding portion that results in one or more of effects (1)-(10) specifically binds to amino acid residues 1-14 of SEQ ID NO: 1 as present in the ecl2 of hSSTR subtype 2. In another embodiment, the anti-hSSTR antibody or antigen-binding portion that results in one or more of effects (1)-(10) specifically binds to amino acid residues 1-14 of SEQ ID NO: 2 in the ecl2 of hSSTR subtype 5.

In one aspect of the invention, contacting an anti-hSSTR antibody of the invention with a biological sample expressing a hSSTR produces a moderately increased level of cAMP compared to the level of cAMP produced by the same biological sample expressing a hSSTR that has been contacted with octreotide.

### Methods of Producing Antibodies and Antibody-Producing Cell Lines

Immunization

The antigen used to generate the anti-hSSTR antibodies of the present invention is a portion of ecl2 of a particular SSTR subtype. In one embodiment, the portion of the ecl2 is from hSSTR subtype 2. In one embodiment, the portion of the ecl2 from hSSTR subtype 2 consists of the 14-mer of SEQ ID NO: 1. In another embodiment, the portion of the ecl2 is from hSSTR subtype 5. In one embodiment, the portion of the ecl2 from hSSTR subtype 5 consists of the 14-mer of SEQ ID NO: 2.

The SSTR antigen used to generate antibodies according to the present invention can be expressed on the surface of a cell that expresses or overexpresses SSTR, or the SSTR antigen can be an immunogenic fragment of SSTR expressed on its surface. The SSTR antigen can be a SSTR fusion protein that is expressed on a cell surface, or the SSTR antigen can be soluble or free (i.e., not bound or expressed on a cell surface).

Animals can be immunized to generate antibodies of the present invention by any method known in the art in view of this disclosure. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are known in the art. See, e.g., Harlow and Lane, supra, and U.S. Pat. No. 5,994,619. In one embodiment, a hSSTR antigen according to the present invention is administered with an adjuvant that stimulates the immune response to the antigen. Exemplary adjuvants include Complete or Incomplete Freund's adjuvant, RIBI (muramyl dipeptides) or ISCOM (immunostimulating complexes). A particularly preferred adjuvant is keyhole limpet hemocyanin (KLH). Such adjuvants may protect the polypeptide from rapid dispersal by sequestering it in a local deposit, or they may contain substances that stimulate the host to secrete factors that are chemotactic for macrophages and other components of the immune system. Preferably, the immunization schedule will involve two or more administrations of the polypeptide over several weeks.

After immunization of an animal with a SSTR antigen, antibodies and/or antibody-producing cells can be obtained from the animal by methods known in the art. For example, serum may be used as it is obtained from the animal, or an immunoglobulin fraction may be obtained from the serum, or anti-hSSTR antibodies may be purified from the serum. The IgG fraction can be separated from the serum using standard methods such as plasmaphoresis or adsorption chromatography with IgG-specific adsorbents such as immobilized Protein A. Monoclonal antibodies may also be prepared using standard methodologies in view of this disclosure. A monoclonal antibody produced according to the present invention can bind to the ecl2 of hSSTR with an affinity of at least 1×10⁻⁷ M, 1×10⁻⁸ M, or 1×10⁻⁹ M. The binding affinity and dissociation rate of a monoclonal anti-hSSTR antibody or antigen-binding portion of the invention can be determined by methods known in the art in view of this disclosure. The binding affinity can be measured, e.g., by competitive ELISAs, RIAs or surface plasmon resonance, such as BIAcore. The dissociation rate can be measured by surface plasmon resonance.

The present invention thus provides a method for producing a monoclonal or polyclonal antibody or antigen-binding portion thereof that specifically binds to amino acid residues 1-14 of SEQ ID NO: 1 as present in the ecl2 of hSSTR subtype 2, said method comprising immunizing a mammal with a peptide consisting of amino acid residues 1-14 of SEQ ID NO: 1, and isolating an antibody or an antigen-binding portion thereof that specifically binds to amino acid residues 1-14 of SEQ ID NO: 1 as present in the ecl2 of the hSSTR subtype 2. In another embodiment, the present invention provides a method for producing a monoclonal or polyclonal antibody or antigen-binding portion thereof that specifically binds to amino acid residues 1-14 of SEQ ID NO: 2 in the ecl2 of hSSTR subtype 5, said method comprising immunizing a mammal with a peptide consisting of amino acid residues 1-14 of SEQ ID NO: 2, and isolating an antibody or an antigen-binding portion thereof that specifically binds to amino acid residues 1-14 of SEQ ID NO: 2 as present in the ecl2 of the hSSTR subtype 5.

In some embodiments, where monoclonal antibodies are provided according to the invention, antibody-producing immortalized cell lines are prepared from cells isolated from the immunized animal. After immunization, the animal is sacrificed and lymph node and/or splenic B cells are immortalized. Methods of immortalizing cells include, but are not limited to, transfecting them with oncogenes, infecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells can be screened using hSSTR, a portion thereof, or a cell expressing SSTR. In one embodiment, the initial screening can be performed using ELISA or a radioimmunoassay. An example of ELISA screening is provided in WO 00/37504, incorporated herein by reference.

Anti-hSSTR antibody-producing cells, e.g., hybridomas, can be selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

Class and Subclass of hSSTR Antibodies

An anti-hSSTR antibody can be an IgG, an IgM, an IgE, an IgA or an IgD molecule. The class and subclass of anti-hSSTR antibodies of the present invention can be determined by any method known in the art in view of this disclosure. In general, the class and subclass of an antibody can be determined using antibodies that are specific for a particular class and subclass of antibody. Such antibodies are commercially available. The class and subclass can be determined by ELISA or Western Blot, as well as other techniques known in the art. Alternatively, the class and subclass can be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

The selectivity of the anti-hSSTR antibody for SSTR can be determined using known methods in view of this disclosure. For instance, selectivity can be determined using Western blot, FACS, ELISA or RIA.

Identification of SSTR Epitopes Recognized by an Anti-hSSTR Antibody

Anti-hSSTR antibodies and antigen-binding portions of the present invention are those that bind to an epitope within the ecl2 of hSSTR. In one embodiment, the hSSTR subtype 2 antibody or antigen-binding portion binds to at least one epitope within the amino acid sequence of SEQ ID NO:1. In another embodiment, the hSSTR subtype 5 antibody or antigen-binding portion binds to at least one epitope within the amino acid sequence of SEQ ID NO:2.

The present invention further encompasses antibodies and antigen binding portions thereof that compete for binding with an anti-hSSTR antibody or antigen-binding portion of the present invention. Methods for determining such competition are known in the art. In one embodiment, an anti-hSSTR antibody of the present invention is allowed to bind to SSTR under saturating conditions; and the ability of a test antibody to “compete off” the anti-hSSTR antibody from the SSTR is then measured. If the test antibody can bind to the SSTR without competing off the anti-hSSTR antibody, then the test antibody binds to a different epitope than the anti-hSSTR antibody. However, if the test antibody competes off the anti-hSSTR antibody from the SSTR, then the test antibody binds either to the same epitope, or to an overlapping epitope, or to an epitope that is in close physical proximity to the epitope bound by the anti-hSSTR antibody. Such test antibodies are encompassed by the present invention. This experiment can be performed using ELISA, RIA, FACS or surface plasmon resonance.

Modified Antibodies

The term “modified antibody” includes antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody.

The present invention further contemplates the preparation and use of antigen-binding portions of any of the antibodies prepared according to the present invention. Such antigen-binding portions can be prepared from intact antibody molecules using standard methods known in the art in view of this disclosure.

A fusion antibody or immunoadhesin may be made that comprises all or a portion of an anti-hSSTR antibody or antigen-binding portion of the present invention linked to another polypeptide. The polypeptide may be a therapeutic agent, such as a toxin, growth factor or other regulatory protein, or may be a diagnostic agent or detectable label, such as an enzyme that may be easily visualized, such as horseradish peroxidase.

Bispecific antibodies or antigen-binding fragments can be produced by a variety of methods including fusion of hybridomas or linking of Fab′ fragments. See, e.g., Songsivilai & Lachmann, Clin. Exp. Immunol. 79: 315-321 (1990), Kostelny et al., J. Immunol. 148:1547-1553 (1992). In addition, bispecific antibodies may be formed as “diabodies” or “Janusins.” In some embodiments, the bispecific antibody binds to two different epitopes on the SSTR. In some embodiments, the bispecific antibody has a first heavy chain and a first light chain from monoclonal antibody of the invention, and an additional antibody heavy chain and light chain. In some embodiments, the additional light chain and heavy chain also are from one of the above-identified monoclonal antibodies, but are different from the first heavy and light chains.

In some embodiments, the modified antibodies described above are prepared using one or more of the variable domains or CDR regions from an anti-hSSTR monoclonal antibody provided herein, from an amino acid sequence of said monoclonal antibody, or from a heavy chain or light chain encoded by a nucleic acid sequence encoding said monoclonal antibody.

An anti-hSSTR antibody or antigen-binding portion of the present invention can be derivatized or linked to another molecule (e.g., another peptide or protein). In general, the antibodies or antigen-binding portions thereof can be derivatized such that the hSSTR binding is not affected adversely by the derivatization or labeling. Accordingly, the antibodies and antibody portions of the present invention are intended to include both intact and modified forms of the anti-hSSTR antibodies described herein. For example, an antibody or antibody portion of the present invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a chemotherapeutic agent, a pharmaceutical agent, and/or a protein or peptide that can mediate association of the antibody or antibody portion with another molecule (such as a streptavidin core region or a polyhistidine tag).

An anti-hSSTR antibody or antigen-binding portion of the present invention can be conjugated to a chemotherapeutic agent. Techniques for conjugating such a therapeutic moiety like a chemotherapeutic agent to antibodies are known. See, e.g., Amon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982). When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Chemotherapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody or antigen-binding portion of the present invention can be conjugated to a radioisotope, e.g., radioactive iodine, to generate cytotoxic radiopharmaceuticals for treating a hSSTR-related disorder, such as a cancer.

Another type of derivatized antibody is a detectably labeled antibody. Useful detection agents with which an antibody or antigen-binding portion of the invention may be derivatized include fluorescent compounds, including fluorescein, fluorescein isothiocyanate, rhodamine, 5-dimethylamine-1-napthalenesulfonyl chloride, phycoerytirin, lanthanide phosphors and the like. An antibody can also be labeled with enzymes that are useful for detection, such as horseradish peroxidase, β-galactosidase, luciferase, alkaline phosphatase, glucose oxidase, and labels such as quantum dots, nano/micro spheres, gold and the like. When an antibody is labeled with an enzyme, the presence of the labeled antibody can be detected by adding additional reagents that the enzyme uses to produce a reaction product that can be discerned. For example, when horseradish peroxidase is present, the addition of hydrogen peroxide and diaminobenzidine leads to a colored reaction product which is measurable. An antibody can also be labeled with biotin and detected through indirect measurement of avidin or streptavidin binding. An antibody can alternatively be labeled with a pre-determined polypeptide epitope that can be recognized by a secondary reporter (e.g., leucine zipper pair sequences, binding sites for secondary antibodies, metal binding domains, epitope tags). An antibody can also be labeled with a fluorescent reporter, colorimetric reporter, or a novel conjugate that can serve as a reporter. In some embodiments, labels are attached by spacer arms of various lengths to reduce potential steric hindrance.

An anti-hSSTR antibody can alternatively be labeled with a radiolabeled moiety (such as an amino acid). The radiolabel can be used for either diagnostic or therapeutic purposes. For instance, the radiolabel can be used to detect and localize hSSTR-expressing tumors by x-ray or other diagnostic techniques. Alternatively, the radiolabel can be used therapeutically as a toxin against cancerous cells or tumors. Examples of radiolabels that may be appropriate for such purposes include ³H, ¹⁴C, ¹⁵N, ³⁵S, ⁹⁰Y, ⁹⁹Tc, ¹¹¹In, ¹²⁵I, ¹³¹I, and ¹⁷⁷Lu, and quantum dots, nano/micro spheres, metal particles (i.e., gold or iron) particles and carbon nanotubes among others.

An anti-hSSTR antibody can alternatively be derivatized with a chemical group such as polyethylene glycol (PEG). Such groups are useful to improve the biological characteristics of the antibody, e.g., to increase serum half-life or to increase tissue binding.

### Pharmaceutical Compositions and Methods of Use

The anti-hSSTR antibodies and antigen-binding portions of the present invention can be used to therapeutically stimulate or block the biological activity of hSSTR, and to thereby treat a medical condition caused or mediated by the activity of SSTR. Accordingly, the present invention provides pharmaceutical compositions comprising a pharmaceutically acceptable carrier admixed with a therapeutically effective amount of an anti-hSSTR antibody or antigen-binding portion of the present invention. In one embodiment, the antibody or antigen-binding portion has agonist-like properties on hSSTR. In another embodiment, the antibody or antigen-binding portion has antagonist-like properties on hSSTR.

Pharmaceutical compositions of the present invention can be used to treat tumors or cancers overexpressing hSSTR in patients. In one embodiment, the anti-hSSTR antibody or antigen-binding portion of such pharmaceutical composition binds specifically to hSSTR subtype 2, and can be used to treat tumors or cancers expressing hSSTR subtype 2. In another embodiment, the anti-hSSTR antibody or antigen-binding portion of such pharmaceutical composition binds specifically to hSSTR subtype 5, and can be used to treat tumors or cancers overexpressing hSSTR subtype 5.

Pharmaceutical compositions of the present invention can be used to treat, ameliorate, prevent, or reduce the frequency or severity of a neuroendocrine tumor or carcinoid syndrome, or one or more symptoms thereof. Additionally, pharmaceutical compositions of the present invention can be used to treat any disease or symptoms for which octreotide is a treatment. Diseases that may be treated with an a pharmaceutical composition comprising an antibody or antigen-binding portion of the invention include, but are not limited to, acromegaly, the treatment of diarrhea and flushing episodes associated with carcinoid syndrome, and treatment of diarrhea in patients with vasoactive intestinal peptide-secreting tumors, and severe, refractory diarrhea from other causes. In another embodiment, a pharmaceutical composition comprising an antibody or antigen-binding portion of the invention may also be used for the treatment of prolonged recurrent hypoglycemia after sulfonylurea overdose. In another embodiment, a pharmaceutical composition comprising an antibody or antigen-binding portion of the invention may also be used to treat patients with liver cirrhosis, to stop actively bleeding blood vessels and to decrease variceal hemorrhage. Pharmaceutical compositions of the present invention can also be used to treat, ameliorate, prevent or reduce symptoms that are secondary to the secretion of peptide or non-peptide hormones in neuroendocrine tumors. In one embodiment, the symptoms can be modified by agonists or antagonists of SSTR.

Cancers that may be treated by an anti-hSSTR antibody or antigen-binding portion of the present invention possessing agonist-like and/or antagonist-like properties on hSSTR can involve any biological sample, and include but are not limited to brain, lung, squamous cell, bladder, gastric, pancreatic, breast, head (e.g., brain tumor), neck, liver, renal, ovarian, prostate, colorectal, esophageal, gynecological (e.g., uterine sarcomas, carcinoma of the fallopian tubes, carcinoma of the endometrium, carcinoma of the cervix, carcinoma of the vagina or carcinoma of the vulva), nasopharynx, or neuroendocrine (e.g., cancer of the thyroid, parathyroid or adrenal glands tumors, pituitary adenoma, acromegaly), thyroid cancers, melanomas, lymphomas, leukemias or multiple myelomas. In particular, anti-hSSTR antibodies and antigen-binding portions of the present invention can be used to treat, ameliorate, prevent, or reduce the frequency or severity of neuroendocrine tumors, or one or more symptoms thereof. In another embodiment, anti-hSSTR antibodies and antigen-binding portions of the present invention can be used to treat, ameliorate, prevent, or reduce the frequency or severity of carcinomas of the breast and ovary, or one or more symptoms thereof.

Examples of neuroendocrine tumors that may be treated with anti-hSSTR antibodies or antigen-binding portions of the present invention include adrenal pheochromocytoma, gastrinoma, glucagonoma, insulinoma, medullary carcinoma of the thyroid, multiple endocrine neoplasia syndrome, pancreatic endocrine tumors, paragangliomas, vasoactive intestinal polypeptide tumors, calcitoninoma, neurotensinoma, parathyroid hormone-related peptide tumor, Merkel cell cancer, small-cell lung cancer, neuroblastoma, and Carney's complex. In addition, carcinoid syndrome may be treated with anti-hSSTR antibodies or antigen-binding portions of the present invention.

Antibodies useful in the present invention may comprise a constant region of human IgG (e.g., IgG1, IgG2, or IgG3), which can mediate tumor cell killing by antibody-dependent cell-mediated cytotoxicity (ADCC). Such an antibody can also suppress the growth of tumors that are hSSTR-dependent.

ADCC refers to a cell-mediated reaction in which nonspecific cytotoxic cells that express Fc receptors (FcRs) (e.g. natural killer cells, neutrophils, monocytes, and macrophages) recognize bound antibody on a target cell and subsequently cause destruction such as lysis of the target cell. For example, the Fc receptors recognize the Fc portion of an IgG antibody of the invention, which is bound to the surface of a target cell (e.g., a tumor cell) After the Fc portion of IgG is bound to the Fc receptor on the natural killer cell, the natural killer cell releases factors such as cytokines and cytotoxic granules that are capable of entering the target cell (e.g., a tumor cell) and promoting cell death. Additionally, the natural killer cells, of which the Fc receptor is bound to the Fc portion of the IgG, can also recruit B cells.

To assess ADCC activity of a molecule of interest, an in vitro ADCC assay, such as that described in U.S. Pat. No. 5,500,362 or U.S. Pat. No. 5,821,337 may be performed. For example, the ability of any particular antibody to mediate lysis of the target cell by complement activation and/or ADCC can be assayed. The cells of interest are grown and labeled in vitro; the antibody is added to the cell culture in combination with either serum complement or immune cells which may be activated by the antigen antibody complexes. Cytolysis of the target cells is detected by the release of label from the lysed cells. In fact, antibodies can be screened using the patient's own serum as a source of complement and/or immune cells. The antibody that is capable of activating complement or mediating ADCC in the in vitro test can then be used therapeutically in that particular patient. Useful effector cells for such assays include peripheral blood mononuclear cells and natural killer cells. Alternatively, or additionally, ADCC activity of the molecule of interest may be assessed in vivo, e.g., in a animal model such as that disclosed in Clynes et al. PNAS (USA) 95:652-656 (1998).

As used herein, the term “pharmaceutically acceptable carrier” refers to any and all solvents, excipients, dispersion media, coatings, antibacterial and antifungal agents, isotonic and absorption delaying agents, and the like that are physiologically compatible and can be used to facilitate administration of an active pharmaceutical ingredient, in this case an antibody or antigen-binding portion thereof, to a patient in need of said treatment. Some examples of pharmaceutically acceptable carriers are water, saline, phosphate buffered saline, dextrose, glycerol, ethanol and the like, as well as combinations thereof. In many cases, it will be preferable to include isotonic agents such as, for example, sugars, polyalcohols such as mannitol, sorbitol, or sodium chloride in the composition. Additional examples of pharmaceutically acceptable carriers or components are wetting agents or minor amounts of auxiliary substances such as wetting or emulsifying agents, preservatives or buffers, which enhance the shelf life or effectiveness of the composition.

Pharmaceutical compositions of the present invention may be in any of a variety of forms such as, e.g., liquid, semi-solid or solid dosage forms, including liquid solutions (e.g., injectable and infusible solutions), dispersions or suspensions, tablets, pills, powders, liposomes and suppositories, depending upon the intended mode of administration and therapeutic application. Typical compositions are in the form of injectable or infusible solutions, such as compositions similar to those used for passive immunization of humans. The preferred mode of administration is parenteral (e.g., intravenous, subcutaneous, intraperitoneal or intramuscular). In one embodiment, the pharmaceutical composition is administered by intravenous infusion or injection. In another embodiment, the pharmaceutical composition is administered by intramuscular or subcutaneous injection.

Pharmaceutical compositions will typically be sterile and stable under the conditions of manufacture and storage. The pharmaceutical composition can be formulated as a solution, microemulsion, dispersion, liposome, or other ordered structure suitable to high drug concentration. Sterile injectable solutions can be prepared by incorporating an anti-hSSTR antibody or antigen binding portion thereof in the required amount, in an appropriate solvent with one or more ingredients enumerated herein as required, and followed by filter sterilization. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying to yield a powder of the antibody or antigen binding portion plus any additional desired ingredient from a previously sterile-filtered solution thereof. Prolonged absorption of injectable compositions can be brought about by including in the composition an agent that delays absorption, for example, monostearate salts and gelatin.

In certain embodiments, antibody compositions of the present invention may be prepared with a carrier that serves to protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Such methods for the preparation of such formulations are known in the art. See, e.g., Sustained and Controlled Release Drug Delivery Systems (J. R. Robinson, ed., Marcel Dekker, Inc., New York, 1978).

The antibodies or antigen binding portions thereof of the present invention can be administered by a variety of methods known in the art, although for many therapeutic applications the preferred route/mode of administration will be subcutaneous, intramuscular, or intravenous infusion. Under certain circumstances, the antibody or antigen-binding portion can be administered continuously via minipump. As will be appreciated by the skilled artisan, the route and/or mode of administration will vary depending upon the desired results.

Formulations comprising hSSTR antibodies or antigen-binding portions of the present invention may conveniently be presented in unit dosage form and may be prepared by any methods known in the pharmaceutical arts. See, e.g., Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, N.Y.; Lieberman et al. (eds.) (1990) Pharmaceutical Dosage Forms Tablets Dekker, N.Y.; and Lieberman et al. (eds.) (1990), Pharmaceutical Dosage Forms: Disperse Systems Dekker, N.Y.

Therapeutic formulations may be administered in many conventional dosage formulations. Formulations typically comprise at least one active ingredient, together with one or more pharmaceutically acceptable carriers. Formulations may include those suitable for oral, rectal, nasal, mucosal, transdermal, vaginal or parenteral (including subcutaneous, intramuscular, intravenous and intradermal) administration.

In one embodiment, the present invention provides a method for treating cancer in a patient, comprising: (a) identifying a patient having cancer cells express a hSSTR; and (b) administering to the patient a therapeutically effective amount of an anti-hSSTR antibody or antigen-binding portion of the present invention. In a preferred embodiment, the patient having cancer cells overexpresses hSSTR subtype 2, hSSTR subtype 5 or both hSSTR subtype 2 and hSSTR subtype 5. In one embodiment, the cancer cells express a specific hSSTR subtype, and the antibody or antigen-binding portion specifically binds to that subtype. In another embodiment, the antibody or antigen-binding portion that specifically binds to the particular hSSTR subtype also binds selectively to that hSSTR subtype.

In a particular embodiment, the present invention provides a method for treating cancer in a patient, comprising: (a) identifying a patient having cancer cells overexpressing a human SSTR subtype 2; and (b) administering to the patient a therapeutically effective amount of an anti-hSSTR subtype 2 antibody or antigen-binding portion of the present invention.

In another particular embodiment, the present invention provides a method for treating cancer in a patient, comprising: (a) identifying a patient having cancer cells expressing a human SSTR subtype 5; and (b) administering to the patient a therapeutically effective amount of an anti-hSSTR subtype 5 antibody or antigen-binding portion of the present invention.

In another embodiment, the present invention provides a method for treating carcinoid syndrome in a patient, comprising the steps of: (a) identifying a patient as having carcinoid syndrome; and (b) administering to the patient a therapeutically effective amount of an anti-hSSTR antibody or antigen-binding portion of the present invention.

In another embodiment, the present invention provides a method for treating carcinoid syndrome in a patient, which comprises: (a) identifying a patient having carcinoid syndrome; and (b) administering to the patient a therapeutically effective amount of an anti-hSSTR subtype 2 antibody or antigen-binding portion of the present invention.

In another embodiment, the present invention provides a method for treating carcinoid syndrome in a patient, which comprises: (a) identifying a patient having carcinoid syndrome; and (b) administering to the patient a therapeutically effective amount of a hSSTR subtype 5 antibody or antigen-binding portion of the present invention.

In a further embodiment, the therapeutic methods of the invention further comprise co-administering to the patient a second therapeutic agent useful to treat the same or different indication. The second therapeutic agent can be a chemotherapeutic agent. In some embodiments, the chemotherapeutic agent is selected from the group consisting of: antibodies that specifically bind to targets other than an SSTR; mitotic inhibitors; alkylating agents; anti-metabolites; intercalating antibiotics; growth factor inhibitors; cell cycle inhibitors; enzymes; topoisomerase inhibitors; biological response modifiers; anti-hormones, e.g. anti-androgens; and anti-angiogenic agents. For example, the chemotherapeutic agent can be an antibody that specifically binds to a growth factor or a cytokine, or a cell surface receptor, or the chemotherapeutic agent can be an antineoplastic agent, an anti-angiogenic agent, an antitumor agent, a peptide analogue that activates SSTR, a soluble SST, a chemical agent that activates SSTR, or an anti-emetic agent. Alternatively, the second therapeutic agent can be radiation treatment.

The second therapeutic agent may be included in the same composition as the anti-hSSTR antibody or antigen-binding portion of the present invention, or it may be co-administered in a separate formulation. Such combination therapy may provide a benefit of lowering the effective dosage of the anti-hSSTR antibody or antigen-binding portion of the present invention, and/or the effective dosage of the co-administered second therapeutic agent, (e.g., by providing a synergistic effect between the two agents), and thereby reducing the potential of possible toxicities or complications associated with either or both of the agents.

Anti-angiogenic agents include MMP-2 (matrix-metalloproteinase 2) inhibitors, MMP-9 (matrix-metalloproteinase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in the art including, e.g., in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference.

An antibody of antigen-binding portion of the present invention can also be used with a signal transduction inhibitor, such as an agent that can inhibit EGF-R (epidermal growth factor receptor) responses, such as EGF-R antibodies, EGF antibodies, and molecules that are EGF-R inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, trastuzumab (HERCEPTIN™ Genentech, Inc.). EGF-R inhibitors are described, for example, in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated), ABX-EGF (Abgenix/Cell Genesys), EMD-7200 (Merck KgaA), EMD-5590 (Merck KgaA), MDX-447/H-477 (Medarex Inc. and Merck KgaA), and the compounds ZD-1834, ZD-1838 and ZD-1839 (AstraZeneca), PKI-166 (Novartis), PKI-166/CGP-75166 (Novartis), PTK 787 (Novartis), CP 701 (Cephalon), leflunomide (Pharmacia/Sugen), CI-1033 (Warner Lambert Parke Davis), CI-1033/PD 183,805 (Warner Lambert Parke Davis), CL-387,785 (Wyeth-Ayerst), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co.), VRCTC-310 (Ventech Research), EGF fusion toxin (Seragen Inc.), DAB-389 (Seragen/Lilgand), ZM-252808 (Imperial Cancer Research Fund), RG-50864 (INSERM), LFM-A12 (Parker Hughes Cancer Center), WHI-P97 (Parker Hughes Cancer Center), GW-282974 (Glaxo), KT-8391 (Kyowa Hakko) and EGF-R Vaccine (York Medical/Centro de Immunologia Molecular (CIM)). These and other EGF-R-inhibiting agents can be used in the present invention.

VEGF inhibitors, for example SU-5416 and SU-6668 (Sugen Inc.), SH-268 (Schering), and NX-1838 (NeXstar) can also be combined with an antibody or antigen-binding portion of the present invention. VEGF inhibitors are described, for example, in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of specific VEGF inhibitors that may be useful in the present invention are IM862 (Cytran Inc.); anti-VEGF monoclonal antibody of Genentech, Inc.; and angiozyme (a synthetic ribozyme from Ribozyme and Chiron). These and other VEGF inhibitors can be used in the present invention as described herein. ErbB2 receptor inhibitors, such as GW-282974 (Glaxo Wellcome plc), and the monoclonal antibodies AR-209 (Aronex Pharmaceuticals Inc.) and 2B-1 (Chiron), can be combined with an antibody or antigen-binding portion of the present invention.

A pharmaceutical composition of the present invention preferably includes a “therapeutically effective amount” of an antibody or antigen-binding portion of the present invention. A “therapeutically effective amount” refers to an amount effective, at dosages and for periods of time necessary, to achieve the desired therapeutic result. In one embodiment, a “therapeutically effective amount” refers to an amount of an anti-hSSTR antibody or antigen-binding portion of the present invention that will result in cancer cell death, arresting or reducing the rate of a cancer cell proliferation, or reducing or ameliorating one or more symptoms of a neuroendocrine tumor or carcinoid syndrome. A therapeutically effective amount of the antibody or antigen-binding portion may vary according to factors such as the disease state, age, sex, and weight of the individual, and the ability of the antibody or antigen-binding portion to elicit a desired response in the patient. A therapeutically effective amount is typically one in which the potential for toxic or detrimental effects of the treatment is outweighed by the therapeutically beneficial effects.

Dosage regimens can be adjusted to provide the optimum desired response (e.g., a therapeutic response). For example, a single bolus can be administered, or several divided doses can be administered over time, or the dose can be proportionally reduced or increased as indicated by the exigencies of the particular therapeutic situation. It is especially advantageous to formulate parenteral compositions in dosage unit form for ease of administration and uniformity of dosage. “Dosage unit form” as used herein refers to physically discrete units suited as unitary dosages for the patient being treated; each unit containing a predetermined quantity of active compound calculated to produce the desired therapeutic effect in association with the required pharmaceutical carrier.

An exemplary, non-limiting range for a therapeutically effective amount of an antibody or antigen-binding portion of the present invention is 0.025 to 50 mg/kg, more preferably 0.1 to 50 mg/kg, more preferably 0.1-25, 0.1 to 10 or 0.1 to 3 mg/kg. Dosage values may vary with the type and severity of the condition to be treated. For any particular patient, specific dosage regimens should be adjusted over time according to the individual need and the professional judgment of the medical practitioner administering or supervising the administration of the composition.

The pharmaceutical composition may be administered, e.g., from three times daily to once every six months. A pharmaceutical composition of the present invention will typically be administered for as long as the cancer is present, provided that administration of the pharmaceutical composition produces positive therapeutic results, such as, e.g., inhibiting or stopping cancer cell proliferation or resulting in cancer regression.

In another aspect, an anti-hSSTR antibody or antigen-binding portion of the present invention can be used therapeutically to induce apoptosis of specific cells in a patient. In many cases, the cells targeted for apoptosis are cancer cells. Thus, the present invention provides a method of inducing apoptosis by administering to a patient in need thereof an amount of an anti-hSSTR antibody or antigen-binding portion of the present invention that is effective at inducing apoptosis.

In another aspect, an anti-hSSTR antibody or antigen-binding portion of the present invenion can be used therapeutically to induce cell cycle arrest of specific cells in a patient. In many cases, the cells targeted for cell cycle arrest are cancer cells. Thus, the present invention provides a method of inducing cell cycle arrest by administering to a patient in need thereof an amount of an anti-hSSTR antibody or antigen-binding portion of the present invention that is effective at inducing cell cycle arrest.

In another aspect, an anti-hSSTR antibody or antigen-binding portion of the present invention can be used therapeutically to suppress neuroendocrine dense core granule content release in a patient. In many cases, the cells targeted for such suppression are cancer cells. Thus, the present invention provides a method of suppressing neuroendocrine dense core granule content release by administering to a patient in need thereof an amount of an anti-hSSTR antibody or antigen-binding portion of the present invention that is effective at suppressing neuroendocrine dense core granule content release.

In another aspect, an anti-hSSTR antibody or antigen-binding portion of the present invention can be used therapeutically to suppress serotonin release in cells of a patient. In many cases, the cells targeted for such suppression are cancer cells. Thus, the present invention provides a method of suppressing serotonin release by administering to a patient in need thereof an effective amount of an anti-hSSTR antibody or antigen-binding portion or the present invention that is effective at suppressing serotonin release.

In another aspect, an anti-hSSTR antibody or antigen-binding portion of the present invention can be used therapeutically to inhibit abnormal cell growth in a patient. In many cases, the cells targeted for such inhibition are cancer cells. Thus, the present invention provides a method of inhibiting abnormal cell growth by administering to a patient in need thereof an effective amount of an anti-hSSTR antibody or antigen-binding portion of the present invention that is effective at inhibiting abnormal cell growth.

The present invention further provides kits comprising an anti-hSSTR antibody or antigen-binding portion of the present invention, or a pharmaceutical composition comprising the same. A kit may further comprise one or more sterile container, and one or more diagnostic agents. A kit may further comprise printed instructions for use of the composition in a diagnostic or therapeutic method of the present invention.

### Diagnostic Methods

The anti-hSSTR antibodies and antigen-binding portions of the present invention can be used to detect hSSTR either in vivo or in vitro in a biological sample. Accordingly, the present invention further provides diagnostic methods.

As used herein, the term “biological sample” means a portion (e.g., a cell or a tissue) taken from a living mammal that is studied or tested as being representative of the whole living tissue or the whole living mammal.

In one embodiment, the present invention provides a method for detecting the presence of a hSSTR in a biological sample, comprising the steps of: contacting the biological sample with an anti-hSSTR antibody or antigen-binding portion of the present invention under conditions permitting the specific binding of the antibody or antigen-binding portion to the hSSTR to form a complex, and detecting the presence of the complex, wherein the presence of the complex indicates the presence of a hSSTR.

In another embodiment, the present invention provides a method for detecting the presence of a hSSTR subtype 2 comprising amino acids 1-14 of SEQ ID NO: 1 in a biological sample, comprising the steps of: contacting the biological sample with an antibody or antigen-binding portion of the present invention under conditions permitting the specific binding of the antibody or antigen-binding portion to the SSTR subtype 2 to form a complex, and detecting the presence of the complex, wherein the presence of the complex indicates the presence of a hSSTR subtype 2.

In another embodiment, the present invention provides a method for detecting the presence of a hSSTR subtype 5 comprising amino acids 1-14 of SEQ ID NO: 2 in a biological sample, comprising the steps of: contacting the biological sample with an antibody or antigen-binding portion thereof of the invention under conditions permitting the specific binding of the antibody or antigen-binding portion to the SSTR subtype 5 to form a complex, and detecting the presence of the complex, wherein the presence of the complex indicates the presence of a hSSTR subtype 5.

The detection methods of the present invention can be carried by use of a conventional immunoassay, including, without limitation, an ELISA, an RIA, FACS, tissue immunohistochemistry, Western blot or immunoprecipitation. In one embodiment, the hSSTR antibody or antigen-binding portion is detectably labeled. In another embodiment, the hSSTR antibody, as the primary antibody, is unlabeled, and a second antibody or other molecule that can bind to the hSSTR antibody is detectably labeled. As known in the art, a secondary antibody is chosen that is able to specifically bind to the primary antibody. For example, if the hSSTR antibody is a human IgG, then the secondary antibody may be an anti-human-IgG antibody. Examples of other molecules that can bind to antibodies include Protein A and Protein G, both of which are available commercially, e.g., from Pierce Chemical Co.

Suitable detectable labels for antibodies and antigen-binding portions have been disclosed supra, and include various enzymes, prosthetic groups, fluorescent materials, luminescent materials and radioactive materials. Examples of suitable enzymes include horseradish peroxidase, alkaline phosphatase, β-galactosidase, and acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride and phycoerythrin. An example of a luminescent material includes luminal. Examples of suitable radioactive labels include ¹²⁵I, ¹³¹I, ³⁵S or ³H.

In other embodiments, hSSTR can be detected and quantified in a biological sample by use of a competition immunoassay utilizing hSSTR standards labeled with a detectable label and an unlabeled anti-hSSTR antibody or antigen-binding portion thereof. In this assay, the biological sample, a labeled hSSTR standard, and the anti-hSSTR antibody or antigen-binding portion are combined, and the amount of labeled hSSTR standard bound to the unlabeled antibody is determined. The amount of hSSTR in the biological sample is inversely proportional to the amount of labeled hSSTR standard bound to the hSSTR antibody.

The immunoassays disclosed above can be used for a number of purposes. For example, anti-hSSTR antibodies can be used to detect hSSTR in in vitro cell cultures. In certain embodiments, the anti-hSSTR antibodies are used to determine the number of hSSTR on the surface of cells to serve as a correlation for the effectiveness of SSTR targeted therapy. This method can be used to identify compounds that are useful to activate or inhibit hSSTR. According to this method, one biological sample is treated with a test compound for a period of time while another biological sample is left untreated. If the total level of hSSTR is to be measured, the cells are lysed and the total hSSTR level is measured using one of the immunoassays described above. The total level of hSSTR in the treated versus the untreated cells is compared to determine the effect of the test compound. In a preferred embodiment, Octreoscan®, which uses a radioactive labeled (Indium 111) octreotide to identify SSTR among neuroendocrine tumors in humans, is used to measure levels of hSSTR in a biological sample. In another preferred embodiment, Western blot analysis is used for detecting SSTR levels in a biological sample.

The anti-hSSTR antibodies of the invention can be used to quantify hSSTR in a biological sample or in cells derived from the biological sample. In some embodiments, the biological is a diseased tissue. In some embodiments, the biological sample is a tumor or a tissue biopsy thereof. The tissue or biopsy is then used in an immunoassay to determine, e.g., total hSSTR levels, cell surface levels of hSSTR or localization of hSSTR by the methods discussed above.

Diagnostic methods described herein can be used to determine whether a tumor expresses high levels of hSSTR, which could be indicative that the tumor is a target for treatment with an anti-hSSTR antibody or antigen-binding portion of the present invention. These diagnostic methods can also be used to monitor the efficacy of any therapeutic treatment directed against the tumor, including any therapeutic treatments provided by the present invention. The diagnostic methods can also be used to determine whether a biological sample comprising a tissue or cell that expresses an insufficient level of hSSTR, and thus is a candidate for treatment with hSSTR antibodies, SST and/or other therapeutic agents for increasing hSSTR levels or activity.

The antibodies of the present invention can also be used in vivo to identify biological samples that express hSSTR. In some embodiments, the anti-hSSTR antibodies or antigen-binding portions of the present invention are used to identify hSSTR-expressing tumors.

The method comprises the steps of administering a detectably labeled anti-hSSTR antibody or antigen-binding portion thereof to a patient in need of a diagnostic test for SSTR, and subjecting the patient to an appropriate type of imaging analysis to identify and localize and SSTR-expressing biological samples. Appropriate imaging analysis is known in the medical arts, and examples include x-ray analysis, magnetic resonance imaging (MRI), and computed tomography (CE). The anti-hSSTR antibody or antigen-binding portion thereof can be labeled with any agent that is appropriate for the particular type of in vivo imaging being utilized. Examples include contrast agents such as barium, which can be used for x-ray analysis, and magnetic contrast agents such as a gadolinium chelate, which can be used for MRI or CE. Examples of other labeling agents include radioisotopes, such as ⁹⁹Tc. In another embodiment, the anti-hSSTR antibody or antigen-binding portion thereof will remain unlabeled, but will be indirectly imaged by administration of a secondary antibody or other molecule that is detectable and that can specifically bind the anti-hSSTR antibody or antigen-binding portion thereof.

### Monoclonal Antibodies

If a foreign substance, such as one of the hSSTR peptides described above is injected into a vertebrate such as a mouse or a human, some of the immune system's B-cells will turn into plasma cells and begin producing antibodies that recognize that antigen. Each B-cell produces only one kind of antibody, but different B-cells will produce structurally different antibodies that bind to different parts (“epitopes”) of the injected antigen. This mixture of antibodies is known as polyclonal antibodies.

The methods and compositions described in embodiments of the invention above can be achieved by using monoclonal antibodies, which can be produced either in cell culture or in live animals. For example, various peptides of 14 amino acid residues in length, and derived from the ecl2 of each of hSSTR subtypes 1, 2, 3, 4 and 5 as described above, can be utilized to generate hSSTR subtype-specific monoclonal antibodies. Exemplary antigens that could be used to generate monoclonal antibodies include hSSTR1: TVACNM1MPEPAQR (SEQ ID NO: 3); hSSTR2: QWGRSSCTINWPGE (SEQ ID NO: 1); hSSTR3: PRGMSTCHMQWPEP (SEQ ID NO: 4); hSSTR4: DTRPARGGQAVACN (SEQ ID NO: 5); and hSSTR5: DVQEGGTCNASWPE (SEQ ID NO: 2).

To produce monoclonal antibodies in animals, the B-cells from the spleen or lymph nodes are typically removed from an animal (such as a rabbit or mouse) that has been challenged (e.g., exposed to by injection) several times with the antigen (peptide) of interest. These B-cells are then fused with myeloma tumor cells that can grow indefinitely in culture (myeloma is a B-cell cancer or more specifically a plasmacytoma) and that have lost the ability to produce antibodies. This fusion is accomplished by making the cell membranes more permeable by, for example, the use of polyethylene glycol (PEG) or electroporation. The fused hybrid cells, called hybridomas, will multiply rapidly and indefinitely. The hybridomas are sufficiently diluted to ensure clonality (i.e., to ensure that all cells in the culture stem from the same single cell) and are grown. The antibodies from the different clones are then tested for their ability to bind to the antigen of interest (for example with tests such as ELISA or Antigen Microarray Assay) or immuno-dot blot, and the most sensitive clone is selected. The hybridoma expressing the antibody of interest is then injected into an animal. When the hybridoma is injected into an animal such as mice, the animal produces tumors which secrete an antibody-rich fluid called ascites. Monoclonal antibodies specific to the antigen of interest, such as the various hSSTR subtypes, can then be isolated from the ascites.

The production of recombinant monoclonal antibodies can involve technologies referred to as repertoire cloning or phage display/yeast display. Recombinant antibody engineering can involve the use of viruses or yeast to produce antibodies of interest, rather than animals. These techniques rely on rapid cloning of immunoglobulin (i.e., antibody) gene segments, such as the genes encoding the hSSTR subtypes described above, to create libraries of antibodies from which antibodies with the desired specificities can be selected and isolated. These techniques can be used to enhance the specificity with which antibodies recognize antigens, their stability in various environmental conditions, their therapeutic efficacy, and their detectability in diagnostic applications. Fermentation chambers can be used to produce these antibodies on a large scale.

The production and isolation of monoclonal antibodies is a process that is well-known in the art.

The present invention is further described by way of the following particular examples. However, the use of such examples is illustrative only and is not intended to limit the scope or meaning of the present invention or of any exemplified term. Furthermore, the present invention is not limited to any particular preferred embodiment(s) described herein. Indeed, many modifications and variations of the invention will be apparent to those skilled in the art upon reading this specification, and such “equivalents” can be made without departing from the invention in spirit or scope. The invention is therefore limited only by the terms of the appended claims, along with the full scope of equivalents to which the claims are entitled.

### EXAMPLES

### Example 1

### Production of Polyclonal Antibodies Directed to hSSTR Subtypes

Various peptides of 14 amino acid residues in length, and derived from the ecl2 of each of hSSTR subtypes 1, 2, 3, 4 and 5, were used to generate hSSTR subtype-specific antibodies. The antigens were:

| | | |
|---|---|---|
| hSSTR1: | TVACNMIMPEPAQR; | (SEQ ID NO: 3) |
| hSSTR2: | QWGRSSCTINWPGE | (SEQ ID NO: 1) |
| hSSTR3: | PRGMSTCHMQWPEP | (SEQ ID NO: 4) |
| hSSTR4: | DTRPARGGQAVACN | (SEQ ID NO: 5) |
| and | | |
| hSSTR5: | DVQEGGTCNASWPE | (SEQ ID NO: 2) |

The peptides represented by SEQ ID NOs: 1-5 were conjugated to keyhole limpet hemocyanin (KLH). Lysine was added at the N-terminus of the peptides to couple the peptides to KLH by amino group conjugation using glutaraldehyde. These peptide antigens (50-500 μg) were diluted in 1 ml sterile saline and 1 ml of Complete Freund's Adjuvant (CFA). The peptide antigen and adjuvant were mixed thoroughly to form a stable emulsion. For subsequent preparation of antigen for injection into rabbits, Incomplete Freund's Adjuvant (IFA) was used. IFA was used for the boost injections because it lacks the mycobacteria found in CFA, and so IFA minimizes the side effects. New Zealand white rabbits were immunized by subcutaneous injection with the peptide antigen (50-500 μg/dose/rabbit). The time period between the first injection with a peptide antigen in CFA and the second injection of a peptide antigen in IFA was approximately three weeks. The dose, using IFA, was repeated approximately four times over a thirteen (13) week period. The rabbits were immunized at weeks 3, 6, and 10. At weeks 0, 7, 9, and 11, each rabbit was bled, and the blood collected was centrifuged to obtain pre-immune serum that was saved to monitor the antibody titer during the immunization process and for subsequent testing. In between immunizations, and for the final harvest of the polyclonal antibodies, blood was collected from the medial artery of the rabbit ear with a 21-gauge needle. The whole blood is allowed to set at room temperature for approximately 1 hour before being placed in the refrigerator overnight to allow the clotting process to finalize. The day following the collection of the whole blood, the blood was processed by completion of a 20 minute centrifugation runs at approximately 5,000 rpm followed by an approximately 10 minute second centrifugation run at approximately 5,000 rpm. Following the second centrifugation, the supernatant was collected.

### Example 2

### Anti-hSSTR Subtype 2, Anti-hSSTR Subtype 3, Anti-hSSTR Subtype 4, and Anti-hSSTR Subtype 5 Polyclonal Antibodies of the Present Invention are Subtype Specific

Recombinant proteins with the ecl2 domain from hSSTR subtypes 1, 2, 3, 4 or 5 were attached to the C-terminus of mouse dihydrofolate reductase (mDHFR molecular weight=28 KD). These recombinant proteins were used in Western blots to test subtype specificity for the hSSTR antibodies produced according to Example 1 above. Whole cell lysates from NIH-3T3 were also included in order to test for non-specific binding by the antibodies. Each of four blots was probed with any one of the hSSTR subtype 2, hSSTR subtype 3, hSSTR subtype 4 or hSSTR subtype 5 antibodies. The results of the Western blot experiment showed that all antibodies are subtype specific. The antibody prepared against ecl2 from hSSTR subtype 1 was also tested, but was determined to lack hSSTR subtype 1 affinity because it did not bind to hSSTR-ecl2 subtype 1 recombinant protein (data not shown).

The conclusion of this Example is that anti-hSSTR subtype antibody or antigen-binding portion thereof preferentially binds to the specific subtype for the particular antigen used to generate the antibody or antigen-binding portion thereof. For example, anti-hSSTR subtype 2 antibody or antigen-binding portion thereof specifically binds to ecl2 of hSSTR subtype 2, but does not bind to ecl2 of hSSTR subtype 1, 3, 4, or 5. Anti-hSSTR subtype 3 antibody or antigen-binding portion thereof specifically binds to ecl2 of hSSTR subtype 3, but does not bind to ecl2 of hSSTR subtype 1, 2, 4, or 5. Anti-hSSTR subtype 4 antibody or antigen-binding portion thereof specifically binds to ecl2 of hSSTR subtype 4, but does not bind to ecl2 of hSSTR subtype 1, 2, 3, or 5. Anti-hSSTR subtype 5 antibody or antigen-binding portion thereof specifically binds to ecl2 of hSSTR subtype 5, but does not bind to ecl2 of hSSTR subtype 1, 2, 3, or 4.

### Example 3

### Anti-hSSTR Subtype 2, Anti-hSSTR Subtype 3 and Anti-hSSTR Subtype 5 Antibodies Can Detect Membrane Surface Receptors on Cells

BON cells were used to test the ability of the anti-hSSTR antibodies to recognize endogenous receptors on the surface of a carcinoid cancer cell line. BON cells have been shown to express endogenous hSSTR subtype 1, hSSTR subtype 2, hSSTR subtype 3, and hSSTR subtype 5, but not hSSTR subtype 4 (E. Ludvigsen et al., Med Oncol 21 (3), 285 (2004)). The ability of the antibodies to bind to cell surface receptors can indicate their diagnostic and/or therapeutic potential. BON cells (human carcinoid cells) were seeded, grown and maintained in F12 HAM (Invitrogen, Cat#31765035)/D-MEM (Invitrogen, Cat#10567-014) (50/50) in 10% certified fetal bovine serum (FBS) (Invitrogen, Cat#16000-044). In some of the experiments described herein, 48 hours prior to experimentation BON cells were washed 2 times using 10 ml D-PBS, and maintained in F12 HAM/DMEM cell growth media without 10% FBS.

hSSTR subtype 1 and hSSTR subtype 4 have been reported to have little to no affinity for octreotide (IC₅₀ of 10,000 nM and 1,000 nM, respectively). hSSTR subtype 3 has been reported to have an IC₅₀ of 187±55 nM. hSSTR subtype 2 and hSSTR subtype 5 bind to octreotide with an IC₅₀ of 2.0±0.7 nM and 4.0±0.3 nM, respectively (J. C. Reubi et al., Eur J Nucl Med 27 (3), 273 (2000)).

Flow cytometry and fluorescence microscopy were used in this experiment to detect antibody binding to cell surface receptors. Live cell detection was carried out using a primary antibody (e.g., hSSTR subtype 2 antibody) under conditions allowing it to bind to its antigen; followed by use of a secondary antibody (i.e., an anti-rabbit IgG) conjugated to a fluorophore (e.g., phycoerythrin or quantum dots) under conditions allowing it to bind to the antigen-bound primary antibody. An appropriate light source, 450-485 nm, Qdot 655 (Invitrogen) was used to excite the fluorophore, and the emitted light was detected with a photomultiplier by either a flow cytometry or immunofluorescence microscopy (IFM).

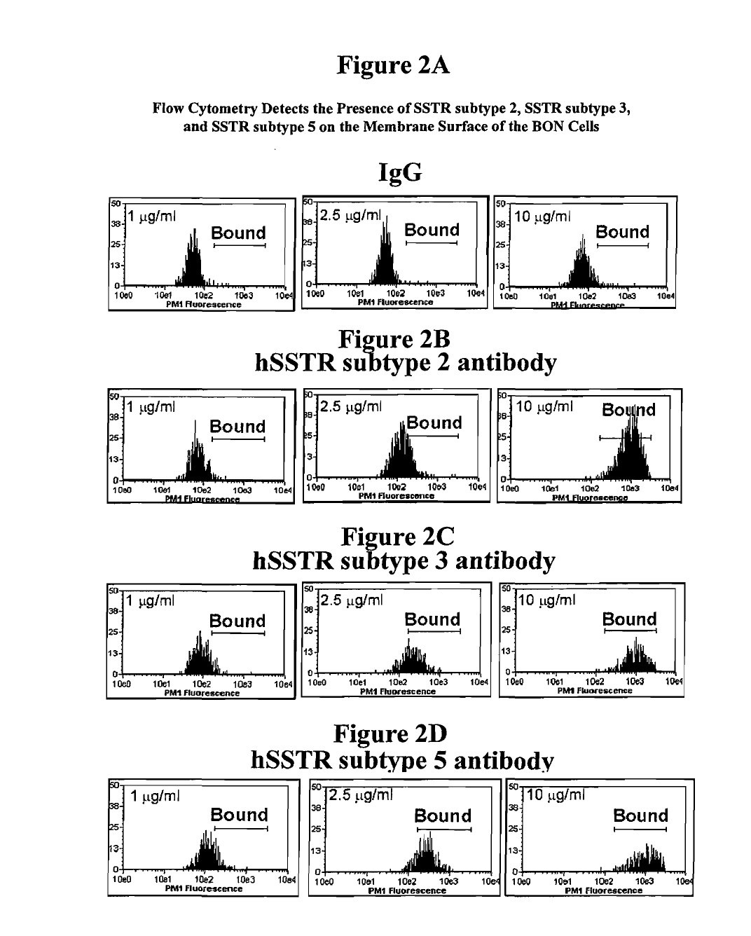

Flow cytometry was used in this experiment to test the binding of anti-hSSTR antibodies to cell surface receptors, and the results are shown in FIG. 2. BON cells were seeded in 6-well tissue culture plates (Costar, Cat#3506). Into each well approximately 200,000 cells/well in 2 ml cell growth media containing 10% FBS (Invitrogen, Cat#16000-044) were plated. The cells were allowed to attach and grow for 48 hours. The cells were collected and counted in accordance with Guava Viacount Flex (Guava Technologies, Cat# 4500-0110) instructions. At the end of each treatment, cells were dissociated from the plate using 1 ml of Cell Dissociation Buffer (Invitrogen Cat#13150016). Dissociated cells were collected in 15 ml Falcon tubes (BD Biosciences, Cat#352099) and 5 ml of cell growth media containing 10% FBS was added. Next, phosphate buffer solution (D-PBS) (Invitrogen, Cat#14190-144) was used as the exchange buffer and the 15 ml Falcon tubes containing test samples were spun using table top centrifugation at 900 rpm (Beckman Coulter). After each centrifugation step, supernatant was discarded and the cell pellet was resuspended using D-PBS with appropriate volume to achieve 300 cells/μl. Next, cells were assayed for hSSTR presence on the cell membrane surface using Guava Express Streptavidin-PE reagent (Guava Technologies, Cat# 4500-0070). Data was collected using Guava PCA96 instrument and analyzed using Guava Express Software Module (Guava Technologies, Cat#0500-0240).

All antibodies, including rabbit IgG, used in this experiment were first tagged with biotin by using ProtOn Biotin Labeling Kit (Vector Laboratories, Cat#PLK-1202), which is required for Guava Express Strepavidin-PE detection. Non-immunized rabbit IgG was used as a control. Anti-hSSTR subtype 2, anti-hSSTR subtype 3, and anti-hSSTR subtype 5 antibodies subtypes were the experimental groups. Each antibody group was treated with three different antibody concentrations, 1 μg/ml, 2.5 μg/ml and 10 μg/ml. The histogram in FIG. 2 is marked to indicate the bound cell population. As the bound cell population increased, the histogram shifted to the right as the concentration of anti-hSSTR subtype 2 antibody, anti-hSSTR subtype 3 antibody or anti-hSSTR subtype 5 antibody added in each experiment increased.

In order to observe anti-hSSTR antibody binding to hSSTR subtype 2 or hSSTR subtype 5 on the membrane surface of the cells, bright field microscopy (using either phase contrast or Nomarski optics) was used to visualize the cells, followed by IFM. Quantum dots (Invitrogen), a type of fluorophore, were conjugated to the secondary antibody Qdot® 655 goat anti-rabbit IgG conjugates (catalogue #Q11421MP). Cells were subjected to immunofluorescence microscopy (IFM) for the detection of hSSTR on the membrane surface of live cells. This was done using our anti-hSSTR antibodies of the invention as the primary antibody. Immediately after cell fixation using 3.7% formaldehyde at 37° C. for 10 minutes, the secondary antibody Qdot® 655 goat rabbit IgG (Invitrogen) was added (1:50) for visualization using the pseudo confocal fluorescent microscope—Zeiss® Axiovert 200M with Apotome noise reduction. The microscope oil lens objective was set at 63×/1.4 for imaging. All fluorescent images captured for Qdot® 655 fluorescence were captured using Cy 3.5 filter with exposure time of 2.5 seconds and using Axiovision 4.4 software for settings and image processing, and these labeled antibodies were used to detect the presence of the primary antibody. The fluorescent color outlining the shape of cells indicated that the anti-hSSTR subtype 2 and anti-hSSTR subtype 5 antibodies were capable of detecting receptors on the surface of BON cells. Both anti-hSSTR subtype 2 antibody and anti-hSSTR subtype 5 antibody can be blocked from binding to the BON cell surface by adding to the incubation mixture the subtype specific ecl2 peptide, which was used to generate antibodies of the invention and that additionally has a lysine at the N-terminus of the peptide, that is specific to hSSTR subtype 2 or hSSTR subtype 5, respectively, that was used to generate each respective antibody. The bright field images were identical to the fluorescence images taken using IFM, indicating that the fluorescence measured is on the same BON cells visible in the bright field image. This experiment showed that both hSSTR subtype 2 and hSSTR subtype 5 antibodies demonstrate subtype binding specificity to BON cells.

IFM was used to test the cross reactivity of the antibodies in CHO—K1 cells (hamster ovary cell line; ATCC, catalogue # CCL-61). Specifically, hSSTR subtype 2 and hSSTR subtype 5 antibodies were tested in CHO—K1 cells were they grown and maintained in Ham's F12 medium F12 HAM (Invitrogen, Cat#31765035) with 10% FBS (Invitrogen, Cat#16000-044) at 37° C. in 95% air and 5% CO₂ incubator. The antibodies were in phosphate-Buffered Saline (D-PBS) (Invitrogen Cat#14190-144) (0.2M monobasic sodium phosphate, 0.2M dibasic sodium phosphate (anhydrous) with 50 mM of NaCl at pH 7.2). Additionally, BON cells were imaged using either anti-hSSTR subtype 2 or anti-hSSTR subtype 5 antibodies. Anti-hSSTR subtype 4 antibodies were used as a negative binding control for the BON cells (since BON cells do not express hSSTR subtype 4). A population of receptor-bound antibodies appeared to be internalized into the cytoplasm within minutes of application.

The conclusion reached based upon the experiments of Example 3 is that anti-hSSTR subtype 2, anti-hSSTR subtype 3, and anti-hSSTR subtype 5 antibodies detected membrane surface hSSTR on BON cells.

### Example 4

### Anti-hSSTR Subtype 2, Anti-hSSTR Subtype 3 and Anti-hSSTR Subtype 5 Antibodies Suppress Cell Growth

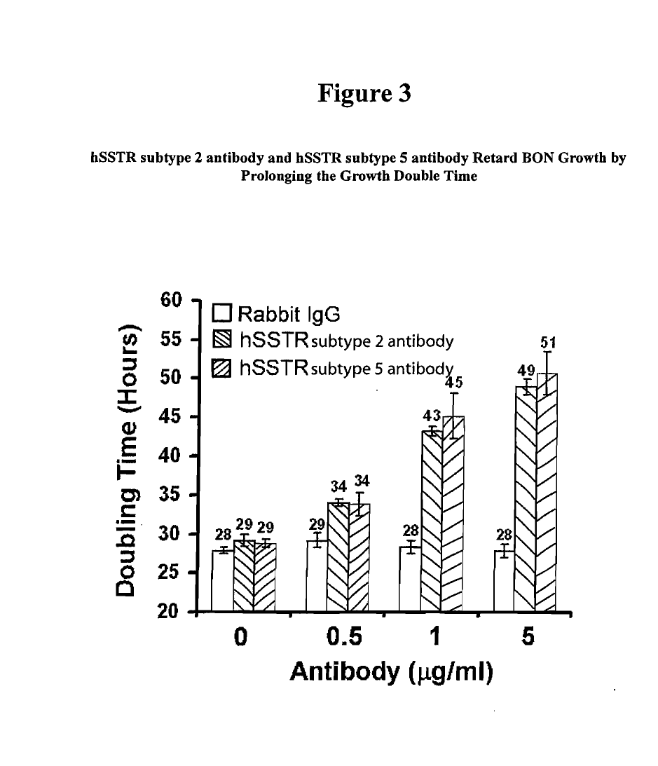

The effects of anti-hSSTR subtype 2 and anti-hSSTR subtype 5 antibodies on cell proliferation were tested using CellTiter 96® Aqueous One Solution Cell Proliferation Assay (MTS) (Promega, Cat#G3581). The assay was performed to measure the growth doubling time for BON cells in vitro (FIG. 3, growth doubling time in hours is presented on the Y-axis). The growth doubling time for BON cells following treatment with rabbit IgG was around 28 to 30 hours (similar to vehicle alone). Cell treatments with anti-hSSTR subtype 2 antibodies or anti-SSTR subtype 5 antibodies were at concentrations of 0 (i.e., untreated), 0.5, 1, and 5 μg/ml (X-axis), and had doubling times as long as 50 hours in a dose-dependent manner (FIG. 3). BON cells were seeded, grown and maintained in F12 HAM (Invitrogen, Cat#31765035)/D-MEM (Invitrogen, Cat#10567-014) (50/50) with 10% certified fetal bovine serum (FBS) (Invitrogen, Cat# 16000-044) in 96-well cell culture dishes (Costar, Cat# 3590) at a density of 2500 cells/well. Cells were allowed to grow for the first 48 hours in 100 μl cell growth media, then when the cells reached a density of approximately 5000 cells/well, the media was removed by aspiration and rinsed with D-PBS) (Invitrogen Cat#14190-144) (0.2M monobasic sodium phosphate, 0.2M dibasic sodium phosphate (anhydrous) with 50 mM of NaCl at pH 7.2). BON cell growth media without FBS was added to each well, and the cells were allowed to grow for another 24 hours before treatment. Cells underwent treatment once every 24 hours. Cell growth was detected at 490 nm (colorimetric detection) by using instrument Victor 3V (Perkin Elmer) once every 24 hours in the duration of 96 hours.

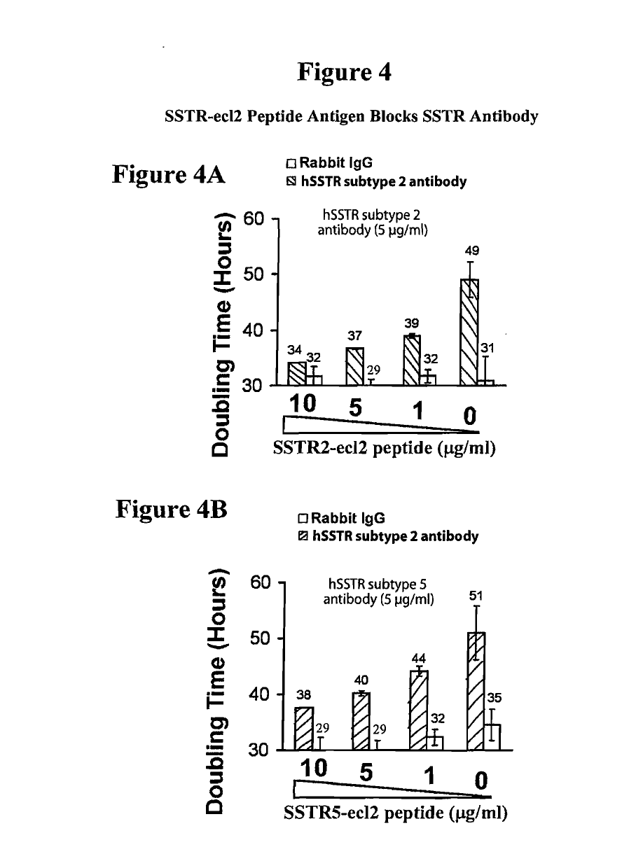

The specificity of antibody inhibition was tested in the MTS assay by using specific antigenic peptides to block the antibodies from binding to the receptor. FIG. 4 shows that ecl2 peptides, which were used to generate antibodies of the invention and that additionally have a lysine at the N-terminus of each peptide can rescue BON cells from anti-hSSTR subtype 2 (FIG. 4A) and anti-hSSTR subtype 5 (FIG. 4B) antibody-induced growth suppression. The Y-axis of FIG. 4 shows the doubling time in the presence or in the absence of anti-hSSTR antibodies, and also the rescue observed when ecl2 peptides, which were used to generate antibodies of the invention and that additionally have a lysine at the N-terminus of each peptide were added in a range from 0, 1, 5 to 10 μg/ml (FIG. 4, X-axis). hSSTR ecl2 peptides, which were used to generate antibodies of the invention and that additionally have a lysine at the N-terminus of each peptide were used to pre-absorb the antibodies, and consequently to alleviate their negative growth effects. As the peptide amount increased, the effect on growth suppression induced by either the anti-hSSTR subtype 2 antibodies (FIG. 4A) or anti-hSSTR subtype 5 antibodies (FIG. 4B) was diminished (FIG. 4, the anti-hSSTR subtype 2 antibody-treated samples are represented by downward diagonal lines; the anti-hSSTR subtype 5 antibody-treated samples are represented by upward diagonal lines; and white bars are negative controls treated with rabbit IgG).

To validate that cell growth inhibition is caused by the anti-hSSTR antibody in a subtype specific manner, BON cells were stably transfected with plasmids carrying short hairpin RNA shSSTR subtype 2 or shSSTR subtype 5 in which the expression of the shRNAs were under the control of tetracycline. Sh-hSSTR subtype 2 and sh-hSSTR subtype 5 destabilize the target receptor's messenger RNA and, consequently, knock down the expression of the target gene. In the presence of tetracycline, the hSSTR subtype 2 and hSSTR subtype 5 mRNA significantly decreased.

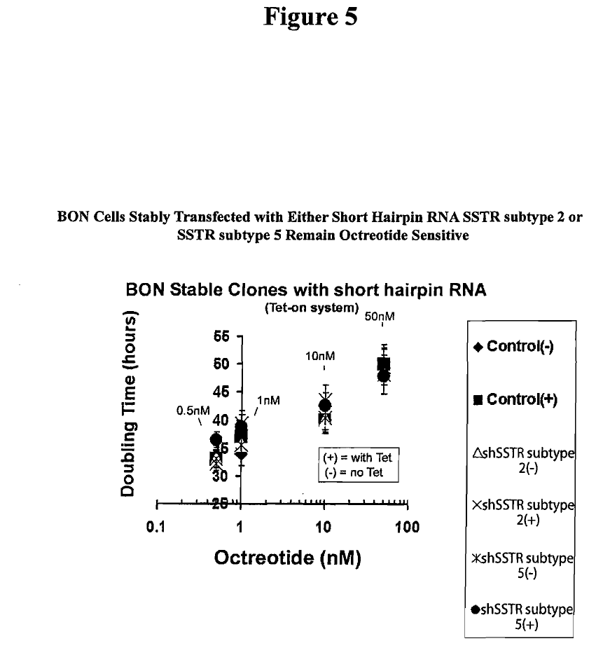

FIG. 5 shows that the growth doubling time in BON cells stably transfected with short hairpin SSTR subtype 2 (sh-hSSTR subtype 2-BON) or with short hairpin SSTR subtype 5 (sh-hSSTR subtype 5-BON) did not change compared to the growth doubling time in BON cells treated with octreotide. The approximate EC₅₀ for octreotide treated BON cells was in the single nM concentrations, which was comparable to the published value (N. Benali et al., Digestion 62 Suppl 1, 27 (2000)). This implies that knocking down only one of the two octreotide-binding receptors (hSSTR subtype 2 or hSSTR subtype 5) does not affect the growth sensitivity of BON cells to octreotide (from 0.5, 1, 10 to 50 nM in concentration). This in turn suggests that the presence of either hSSTR subtype 2 or hSSTR subtype 5 is sufficient to provide the growth inhibition elicited by octreotide.

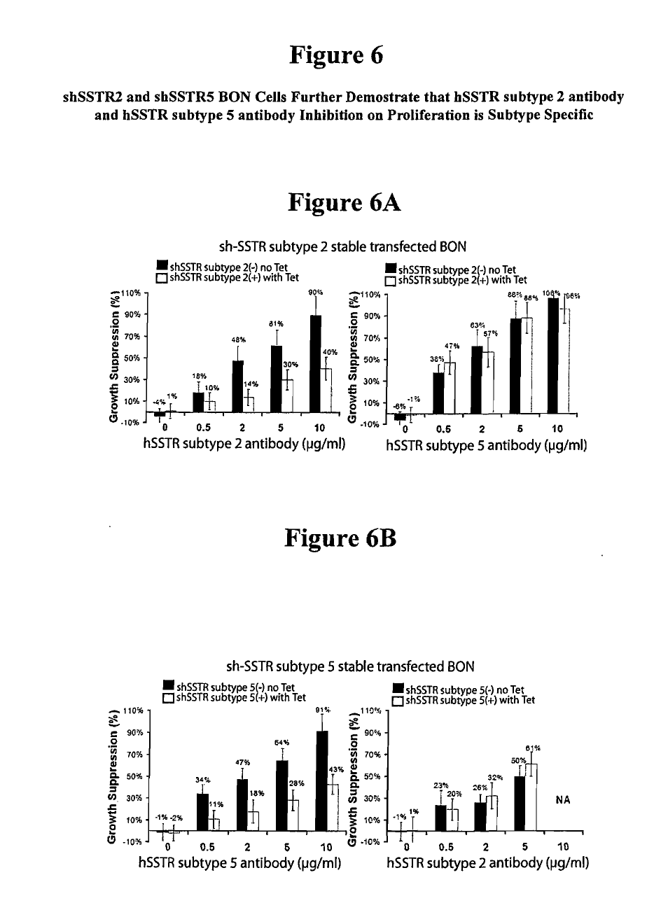

In FIG. 6, the Y-axis represents the calculated value for growth suppression in percentage by applying the difference between the doubling time recorded under saturating octreotide (50 nM) treated samples (doubling time of ˜50 hours) versus untreated samples (doubling time of ˜30 hours). The resulting maximal suppression in hours (20 hours) is designated as 100% of growth suppression. The difference measured in doubling time detected between experimental samples and untreated samples divided by the maximal suppression yields the percent growth suppression.

shSSTR BON cells were used to test the specificity of anti-hSSTR subtype 2 and anti-hSSTR subtype 5 antibodies in suppressing BON cell growth. FIG. 6 shows that all BON cells were separated into two major treatment groups, i.e., the group with tetracycline, and the group without tetracycline. FIG. 6A shows an experiment designed to test the growth inhibition subtype specificity of anti-hSSTR subtype 2 antibodies. FIG. 6B shows an experiment designed to test the growth inhibition subtype specificity of anti-hSSTR subtype 5 antibodies. The left panels of FIG. 6a and FIG. 6B are the experimental group, while the right panels are the negative control. The Y-axis of each of FIG. 6A and FIG. 6B indicates the growth suppression outcome in %, while the X-axis of each of FIG. 6A and FIG. 6B indicates the increasing concentrations of the antibodies added in each sample.

shSSTR BON cells were grown and maintained in F12 HAM/D-MEM in 10% FBS supplemented with 5 μg/ml puromycin. shSSTR 2 or 5 BON cells contain a recombinant shSSTR plasmid pSuperior plasmid (OligoEngine) and, upon addition of 2 μg/ml tetracycline, hSSTR 2 or 5 is down regulated, thus rendering shSSTR BON cells insensitive to the anti-hSSTR antibody treatment, allowing for the determination of anti-SSTR antibody specificity with the experimental detail described here.

FIG. 6A shows that in shSSTR subtype 2-BON cells, the hSSTR subtype 2 antibody's growth suppressive effect was dependent on the presence of hSSTR subtype 2 in BON cells. Because the expression of hSSTR subtype 2 in shSSTR subtype 2-BON cells was down-regulated in the presence of tetracycline, the BON cells became significantly less sensitive to the anti-hSSTR subtype 2 antibodies. Sh-hSSTR subtype 2-BON growth suppression imposed by the hSSTR subtype 5 antibody was the same in the presence or absence of tetracycline and, therefore, was independent of hSSTR subtype 2 expressions. FIG. 6B shows the results of testing for anti-hSSTR5 antibody subtype specificity. In summary, this experiment confirmed that both hSSTR subtype 2 and hSSTR subtype 5 antibodies work in a subtype-specific manner when tested using sh-hSSTR-BON cells.

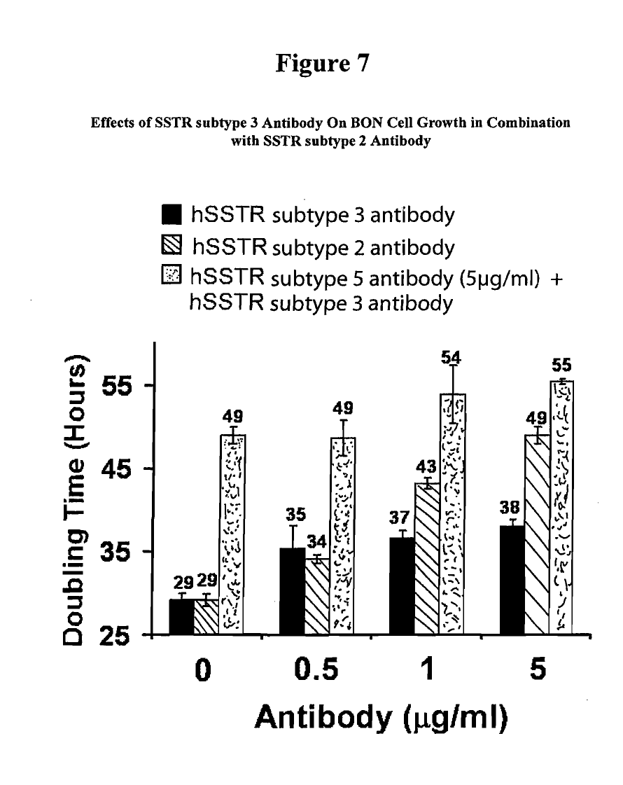

Anti-hSSTR subtype 3 antibodies were shown to impose moderate growth suppression in BON cells FIG. 7 shows that hSSTR subtype 3 antibodies independently imposed growth suppression, and had an additive effect when combined with anti-hSSTR subtype 2 antibody (FIG. 7), but not when combined with anti-hSSTR subtype 5 (data not shown). As anti-hSSTR subtype 3 antibody treatment (0, 0.5, 1 and 5 μg/ml) increased (shown on the X-axis), the doubling time measured in BON cells increased from 29 hours (at 0 μg/ml) up to 38 hours (at 5 μg/ml) in a concentration-dependent manner. For the anti-hSSTR subtype 2 antibodies, the doubling time increased from 29 hours (at 0 μg/ml) to 49 hours (at 5 μg/ml). When anti-hSSTR subtype 2 antibodies and hSSTR subtype 3 antibodies were added together, the growth suppression surpassed that of any one antibody treatment alone, and the effect appeared additive at 1 μg/ml and 5 μg/ml of antibody treatments with the doubling time increasing to approximately 57 hours.

BON cells were seeded, grown and maintained in F12 HAM (Invitrogen, Cat#31765035)/D-MEM (Invitrogen, Cat#10567-014) (50/50) with 10% certified fetal bovine serum (FBS) (Invitrogen, Cat#16000-044) in 96-well cell culture dishes (Costar, Cat# 3590) at a density of 2500 cells/well. Cells were allowed to grow for the first 48 hours in 100 μl cell growth media, then when the cells reached a density of approximately 5000 cells/well, the media was removed by aspiration and rinsed with D-PBS) (Invitrogen Cat#14190-144) (0.2M monobasic sodium phosphate, 0.2M dibasic sodium phosphate (anhydrous) with 50 mM of NaCl at pH 7.2). BON cell growth media without FBS was added to each well, and the cells were allowed to grow for another 24 hours before treatment. Cells underwent antibody treatment once every 24 hours. Cell growth was detected at 490 nm (colorimetric detection) by using instrument Victor 3V (Perkin Elmer) once every 24 hours in the duration of 96 hours.

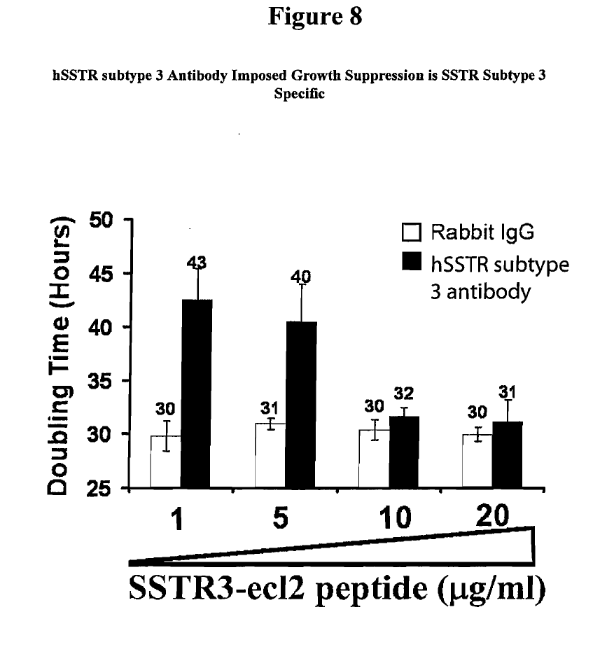

FIG. 8 shows that pre-absorption of the hSSTR subtype 3 antibody with hSSTR3 subtype 3-ecl2 peptide, which was used to generate antibodies of the invention and that additionally has a lysine at the N-terminus of each peptide, reversed the effect of the anti-hSSTR subtype 3 antibody. The addition of hSSTR subtype 3-ecl2 peptide, which was used to generate antibodies of the invention and that additionally has a lysine at the N-terminus of each peptide, did not have any effect on control cells treated with rabbit IgG.

Taken together, the data shown in FIGS. 3-8 from experiments performed with antibodies of the invention show that anti-hSSTR subtype 2, anti-hSSTR subtype 3, and anti-hSSTR subtype 5 antibodies suppressed cell growth.

### Example 5

### Effects of Anti-hSSTR Subtype 2, Anti-hSSTR Subtype 3 and Anti-hSSTR Subtype 5 Antibodies on Cellular Growth Suppression: Death, Apoptosis and Cell Cycle Arrest

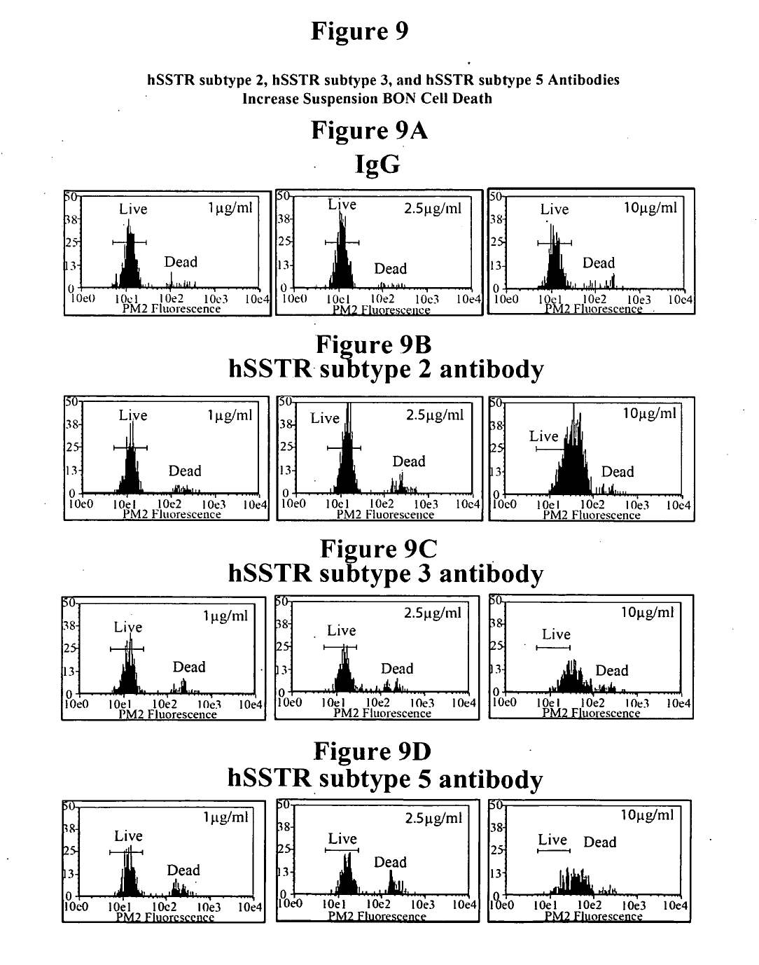

Anti-hSSTR subtype 2, anti-hSSTR subtype 3 and anti-hSSTR subtype 5 antibodies bind to their respective receptor antigens in a subtype specific manner (see Example 2 above). Anti-hSSTR subtype 2 and anti-hSSTR subtype 5 antibodies bind specifically to hSSTRs on the membrane surface of BON cells (see Example 3 above). The biological consequence of an antibody of the invention binding to the receptor is functional agonism resulting in inhibition of cell growth. FIG. 9 shows flow cytometry data demonstrating the effect of anti-hSSTR subtype 2 (FIG. 9B), anti-hSSTR subtype 3 (FIG. 9C) and anti-hSSTR subtype 5 (FIG. 9D) antibodies on cell death. As shown in FIG. 9, antibody treatments used on the BON cells were 1 μg/ml, 2.5 μg/ml, and 10 μg/ml. As the concentration of the antibodies increased from 1 μg/ml to 2.5 μg/ml, and then to 10 μg/ml, the histogram shifts to the right, indicating increased cell death. FIG. 9 shows an estimation of the dead cells that were detected by staining with 7-Amino-Actinomycin D (7-AAD). The gradual shift in the histogram was observed in response to increasing concentrations of anti-hSSTR subtype 2 antibodies, anti-hSSTR subtype 3 antibodies, and anti-hSSTR subtype 5 antibodies, but was not observed in control samples treated with rabbit IgG (FIG. 9A) or with vehicle (data not shown).

BON cells were seeded in 6-well tissue culture plates (Costar, Cat#3506) and each well started out at a density of approximately 200,000 cells/well in 2 ml cell growth media containing 10% FBS (Invitrogen, Cat#16000-044) to allow cells to attach and grow for 48 hours. Cells were then switched to growth media without FBS and allowed to grow for another 24 hours before applying treatments. Samples were collected and counted in accordance with Guava Technologies' Viacount Flex (Cat#4500-0110) instructions. At the end of each antibody treatment, cells were dissociated from the plate using 1× trypsin-EDTA (Invitrogen Cat#2520). Dissociated cells were collected and trypsin-EDTA was quickly quenched using 10 ml cell media containing 10% FBS collected in 15 ml Falcon tubes (BD Biosciences) and cell samples were spun using table top centrifugation at 900 rpm (Beckman Coulter). Next, phosphate buffer solution (D-PBS) (Invitrogen Cat#14190-144) (Invitrogen) was used as the exchange buffer to resuspend the cell pellet. After each buffer exchange centrifugation (2 times), supernatant was discarded and the cell pellet was resuspended with D-PBS using the appropriate volume to achieve 200 cells per μl and is then used to determine the apoptosis state among treated cell populations by using Guava Nexin Kit (Cat#4500-0010), described in the Guava Nexin kit protocol. Detection was achieved by using Guava PCA96 instrument and data was analysed by using Guava Nexin software module as (Cat#0500-0250).

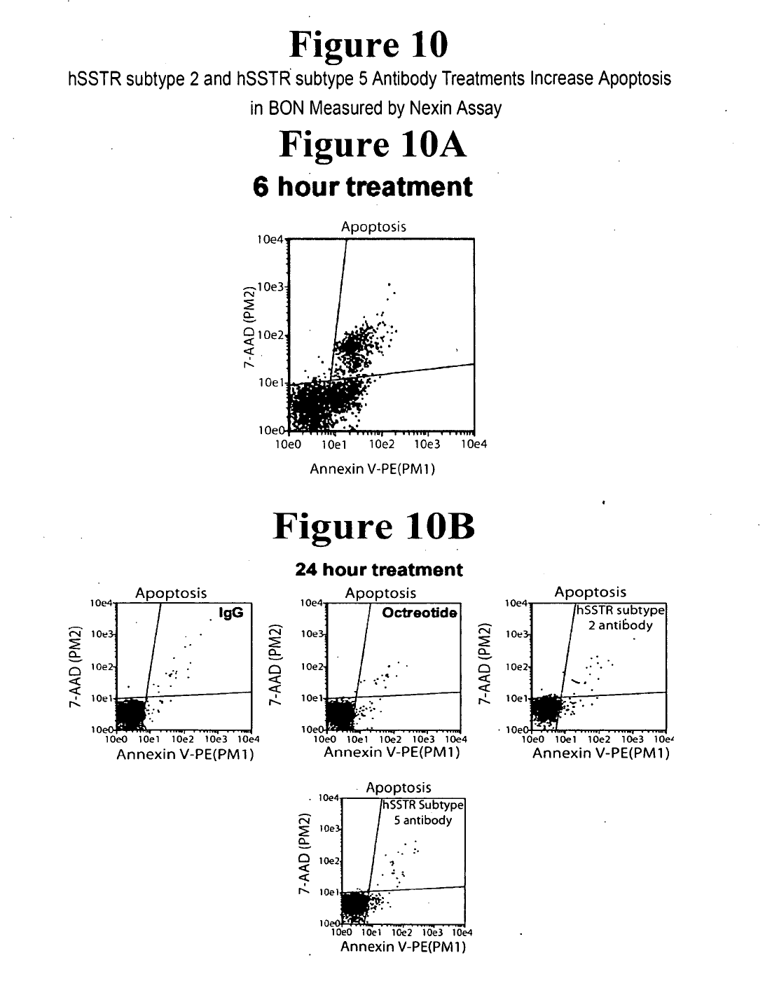

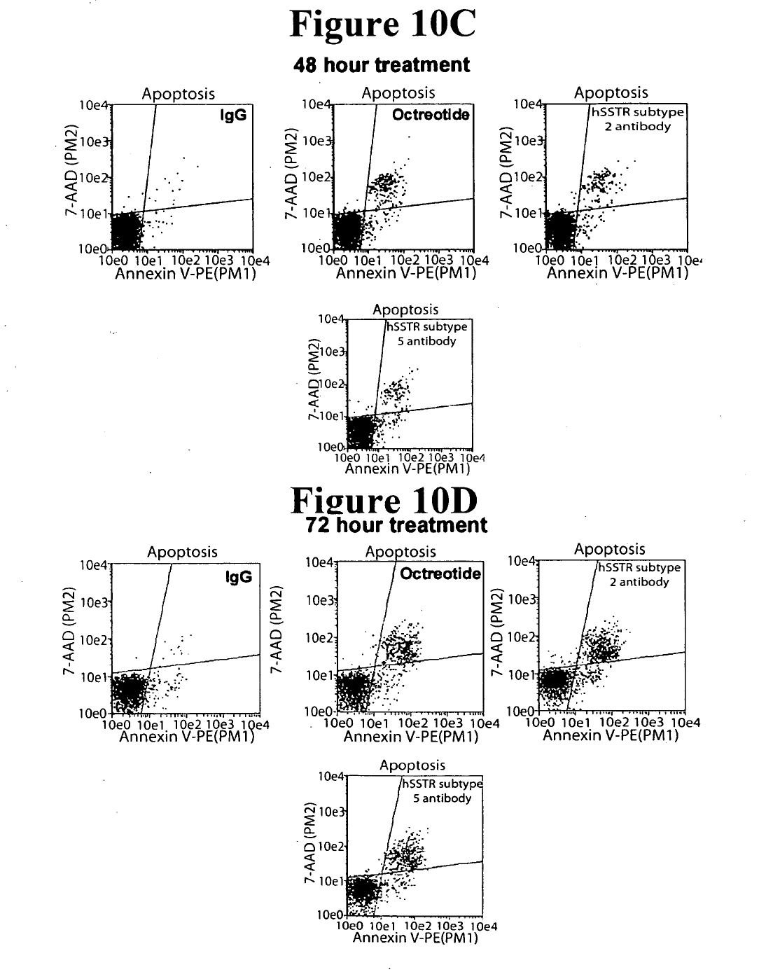

When BON cells were treated with anti-hSSTR subtype 2 or anti-hSSTR subtype 5 antibodies, significant growth suppression was measured (see FIG. 3). This growth suppression may be due to cell death and/or cell cycle arrest. Based upon the observation that anti-hSSTR subtype 2, anti-hSSTR subtype 3, and anti-hSSTR subtype 5 antibodies can increase death in BON cells (see FIG. 9), anti-hSSTR subtype 2, anti-hSSTR subtype 3, and anti-hSSTR subtype 5 antibodies were further tested for their ability to induce apoptosis. As shown in FIG. 10A, BON cells were subjected to 6 hours of staurosporin treatment as a positive control. Following the protocol for the Guava Nexin™ assay (Guava Technologies), two different dyes were used to stain for cells undergoing apoptosis: 7-AAD stains for dead and late apoptosis cells (upper right quadrant of each graph in FIG. 10), and Annexin V stains for cells undergoing apoptosis from the early stage (lower right quadrant of each graph in FIG. 10). BON cells were subjected to either 10 μg/ml rabbit IgG (negative control); or 100 nM octreotide; or 10 μg/ml anti-hSSTR subtype 2 antibodies; or 10 μg/ml anti-hSSTR subtype 5 antibodies. Once every 24 hours for 72 hours (FIGS. 10B, 10C and 10D), cells were collected and tested for apoptosis status using the Guava Nexin™ assay (Guava Technologies). As shown in the Tables and in FIGS. 10B, 10C and 10D, values represent results assayed at each significant increase of apoptosis at the 24-hour time point. The data in FIG. 10 shows samples treated with octreotide; or anti-hSSTR subtype 2 antibodies; or anti-hSSTR subtype 5 antibodies; but not with rabbit IgG. Approximately 10% of the cell population at 48 hours was Annexin V positive, which is indicative of early apoptotic cells. This effect of inducing apoptosis was most apparent around 72 hours at which time approximately 30% of the samples tested Annexin V positive.

TABLE 1A
Quantification of the Apoptosis Results in FIG. 10A at 6 hours
After Treatment with Staurosporin (% of Total)

| | Staurosporine |
|---|---|
| Nuclear debris (upper left | 1.0% |
| Quadrant) | |
| Late apoptotic cells | 23.7% |
| (Upper Right Quadrant) | |
| Live, healthy cells | 46.7% |
| (Lower Left Quadrant) | |
| Early apoptotic cells | 28.7% |
| (Lower Right Quadrant) | |
| Annexin V+ | 52.3% |
| 7-AAD+ | 24.6% |

TABLE 1B
Quantification of the Apoptosis Results in FIG. 10B
at 24 hours after Treatment (% of Total)

| | IgG | Oct. | SSTR2ab | SSTR5ab |
|---|---|---|---|---|
| Nuclear Debris (Upper | 0.1% | 0.1% | 1.5% | 0.4% |
| Left Quadrant) | | | | |
| Late apoptotic cells | 1.1% | 1.0% | 1.1% | 1.2% |
| (Lower Right Quadrant) | | | | |
| Live, healthy cells | 96.8% | 95.7% | 94.8% | 94.4% |
| (Upper Right Quadrant) | | | | |
| Early apoptotic cells | 2.1% | 3.3% | 2.7% | 4.0% |
| (Lower Right Quadrant) | | | | |
| Annexin V+ | 3.1% | 4.3% | 3.8% | 5.2% |
| 7-AAD+ | 1.1% | 1.1% | 2.5% | 1.6% |

TABLE 1C
Quantification of the Apoptosis Results in FIG. 10C
at 48 hours after Treatment (% of Total)

| | IgG | Oct. | SSTR2ab | SSTR5ab |
|---|---|---|---|---|
| Nuclear Debris (Upper | 0.4% | 1.0% | 1.2% | 0.7% |
| Left Quadrant) | | | | |
| Late apoptotic cells | 0.7% | 10.0% | 7.0% | 6.0% |
| (Lower Right Quadrant) | | | | |
| Live, healthy cells | 97.3% | 85.4% | 88.9% | 88.1% |
| (Upper Right Quadrant) | | | | |
| Early apoptotic cells | 1.7% | 3.8% | 2.8% | 5.3% |
| (Lower Right Quadrant) | | | | |
| Annexin V+ | 2.3% | 13.7% | 9.9% | 11.3% |
| 7-AAD+ | 1.1% | 10.9% | 8.3% | 6.7% |

TABLE 1D
Quantification of the Apoptosis Results in FIG. 10D
at 72 hours after Treatment (% of Total)

| | IgG | Oct. | SSTR2ab | SSTR5ab |
|---|---|---|---|---|
| Nuclear Debris (Upper | 0.1 | 0.6% | 1.1% | 1.1% |
| Left Quadrant) | | | | |
| Late apoptotic cells | 0.7% | 19.2% | 25.0% | 18.8% |
| (Lower Right Quadrant) | | | | |
| Live, healthy cells | 94.5% | 72.5% | 67.4% | 74.0% |
| (Upper Right Quadrant) | | | | |
| Early apoptotic cells | 4.8% | 7.7% | 6.5% | 6.1% |
| (Lower Right Quadrant) | | | | |
| Annexin V+ | 5.5% | 26.9% | 31.5% | 25.0% |
| 7-AAD+ | 0.7% | 19.8% | 26.1% | 19.8% |

The pathway contributing to the apoptosis caused by the addition of either anti-hSSTR subtype 2 or anti-hSSTR subtype 5 antibodies was investigated by determining which caspase(s) species was activated and appeared to play a role in anti-hSSTR antibody-induced apoptosis. Caspase 8 expression was studied because it may play a part in sensitizing cells to death ligand induced apoptosis that involves caspase 9 and/or execution caspases, as suggested human pancreatic adenocarcinomas (J. Guillermet et al., Proc Natl Acad Sci USA 100 (1), 155 (2003)). Caspase 8 can induce intracellular acidification activation that will ultimately lead to mitochondrial dysfunction and release of cytochrome c, which are events that typically precede apoptosis (D. Liu et al., J Biol Chem 275 (13), 9244 (2000)).

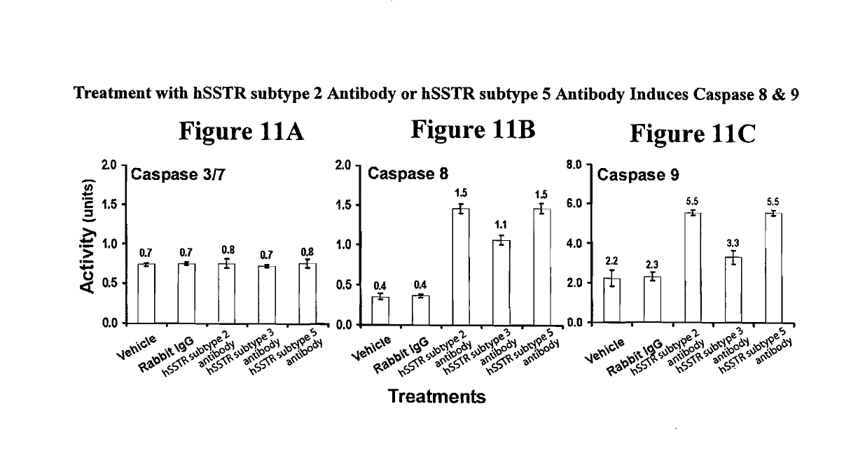

As shown in FIG. 11, caspase activity was measured once every 24 hours for a total of 72 hours. BON cells were treated once every 24 hours with the following treatments: (i) D-PBS (vehicle); (ii) 5 μg/ml rabbit IgG; (iii) 5 μg/ml anti-hSSTR subtype 2 antibodies; (iv) 5 μg/ml anti-hSSTR subtype 3 antibodies; (v) 5 μg/ml anti-hSSTR subtype 4 antibodies; and (vi) 5 μg/ml anti-hSSTR subtype 5 antibodies. The Y-axis of FIG. 11 shows caspase activity. At the 72-hour time point, a significant increase in both caspase 8 (FIG. 11B) and caspase 9 (FIG. 11C) was observed as elicited by treatment with either anti-hSSTR subtype 2 antibodies or anti-hSSTR subtype 5 antibodies. No significant increase in caspase 3 or caspase 7 was observed after treatment with either anti-hSSTR subtype 2 antibodies or anti-hSSTR subtype 5 antibodies (FIG. 11A).

BON cells were plated in full medium at a seeding density of 2,500 cells/well in 96-well white-walled luminometer plates (Perkin Elmer Cat#6005680) for cell culture 48 hours prior to the treatment. BON cells were grown at 37° C. with 95% air and 5% CO₂. On the day of the treatment, full medium was aspirated, washed once with D-PBS and replaced with 100 μl/well serum depleted medium and allowed to equilibrate for 24 hours. Treatments were added in triplicate and assayed using the Promega Caspase-Glo® system (Promega: Caspase-Glo® 3/7 Assay, Cat#G8090; Caspase-Glo® 8 Assay Cat#8200; and Caspase-Glo® 9 Assay, Cat#8210). Standard curves for each caspase assay were facilitated using recombinant proteins of human Caspase-3 (Chemicon Cat#CC119), human Caspase-8 (Chemicon Cat#CC123) and human Caspase-9 (Chemicon Cat#CC120). Each recombinant protein was reconstituted in 25 μl sterile filtered D-PBS containing 15% glycerol in siliconized tubes yielding a stock concentration of 1 unit/μl. For additional details, please see protocol from Promega Caspase-Glo® assay kits (see above).

Cell cycle arrest appeared to play a role in the growth suppression (Examples 4 and 5) observed with anti-hSSTR subtype 2 antibody and anti-hSSTR subtype 5 antibody treatments. Propidium iodide dye was used in flow cytometry (Guava Cell Cycle Reagent Cat #4500-0220) to stain DNA and detect cell cycle progression in BON cells. Treatment conditions for the experiment were as follows: (i) D-PBS; (ii) 5 μg/ml rabbit IgG; (iii) 5 μg/ml anti-hSSTR subtype 2 antibodies; (iv) 5 μg/ml anti-hSSTR subtype 5 antibodies; (v) 100 nM octreotide; and (vi) 2 μM Nocodazole.

0.75 million BON cells were seeded, grown and maintained in F12 HAM (Invitrogen, Cat#31765035)/D-MEM (Invitrogen, Cat#10567-014) (50/50) in 10% certified fetal bovine serum (FBS) (Invitrogen, Cat#16000-044) in 10 cm tissue culture plates (BD Biosciences Cat#353003). 48 hours later, the BON cells were washed 2 times using 10 ml D-PBS and cell growth media without FBS was added to the cells for another 24 hours (grown to about 3 million cells) before treating with anti-hSSTR antibodies. Treatments, as described above, were added to the cells once every 24 hours for a total of 48 hours. At the end of each treatment, cells were dissociated from the plate using 2 ml 1× trypsin-EDTA (Invitrogen Cat#2520). Dissociated cells were collected and transferred to 15 ml Falcon tubes (BD Biosciences, Cat#352099) and trypsin-EDTA was immediately quenched by the addition of 8 ml cell media containing 10% FBS. Next, phosphate buffer solution (D-PBS) (Invitrogen Cat#14190-144) was used as the exchange buffer and 15 ml Falcon tubes with samples were spun at 900 rpm using a table top centrifuge (Beckman Coulter). After centrifugation, supernatant was discarded and cell pellet was resuspended in 10 ml of PBS and this was repeated two times. After the third and the final centrifugation, supernatant was discarded and the cell pellet was resuspended with prechilled 100% methanol (in −20° C. freezer) using the appropriate volume to achieve 1000 cells per μl and stored for 24 hours prior to detection. Guava Cell Cycle Reagent (Guava Technologies, Cat#4500-0220) was used for detection by using the Guava PCA96 instrument. Cell Cycle data was analyzed by using Guava Cell Cycle module (Guava Technologies, Cat# 0500-0480). For further detail, please see Guava Cell Cycle Reagent protocol (Guava Technologies).

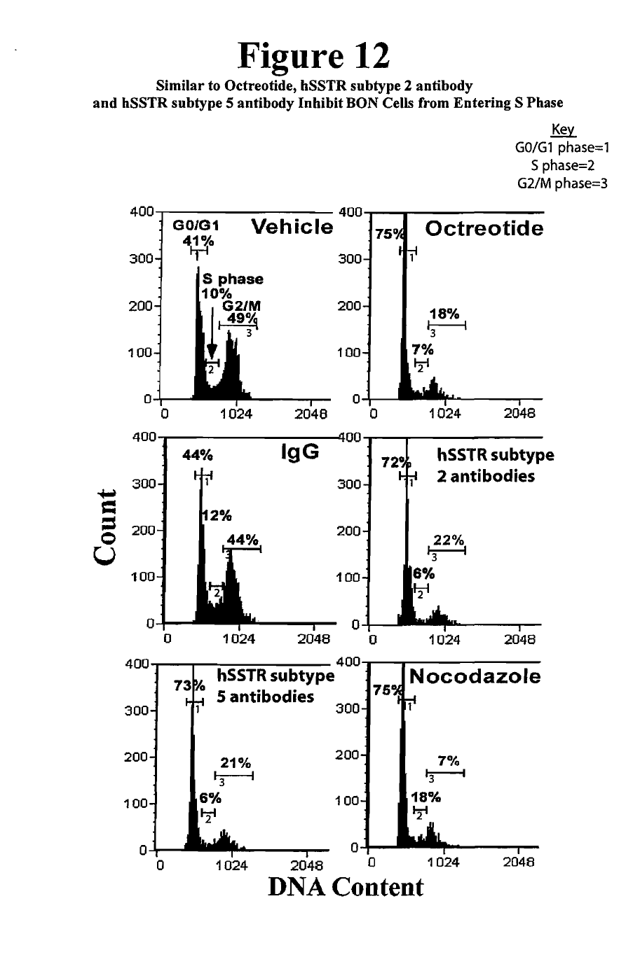

The results are presented in FIG. 12. In negative control treatments, such as vehicle or IgG, the cell populations were evenly distributed between G0/G1 and G2/M phases (˜40 percent plus at each phase), and only ˜10% were in the DNA synthesis (S) phase. In the presence of either the anti-hSSTR subtype 2 antibodies, the anti-hSSTR subtype 5 antibodies, or octreotide, there was a dramatic shift of cell population to G₀/G₁ (increased to ˜75%) compared to cells treated with either vehicle or with rabbit IgG. This was similar to the cell cycle arrest agent Nocodazole (2 μM; Sigma-Aldrich; positive control), as shown in the bottom right corner of FIG. 12F. Thus, anti-hSSTR subtype 2 antibodies and anti-hSSTR subtype 5 antibodies appear to activate each of their respective receptors, and prevent BON cells from entering the S phase. The activation of hSSTR has been reported to activate retinoblastoma proteins, and to keep cells from progressing out of the G1 into the S phase.

Anti-hSSTR subtype 2, anti-hSSTR subtype 3, and anti-hSSTR subtype 5 antibodies were each found to cause cellular growth suppression, and this growth suppression was further determined to be the result of induced cell death, apoptosis and cell cycle arrest (as indicated above). However, hSSTR activation by the antibodies did not seem to negatively affect a second messenger pathway (adenylyl cyclase activity) and with a minor cAMP increase, perhaps by blocking endogenous hSST's ability to inhibit adenylyl cyclase. Thus, these antibodies seem to possess both agonist-like and antagonist-like properties. The experimental evidence provided herein suggests that the anti-hSSTR subtype 2 and anti-hSSTR subtype 5 antibodies work like SSTR agonists.

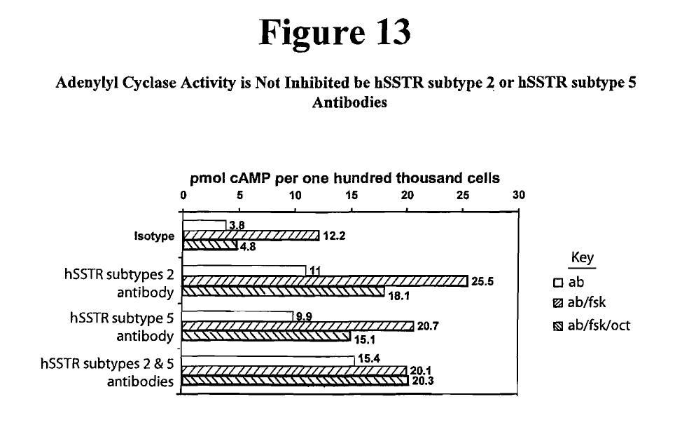

This was examined further by measuring cAMP output, as catalyzed by adenylyl cyclase stimulated by forskolin (fsk). Because SSTR is negatively coupled to adenylyl cyclase, SSTR activation can dampen the output of cAMP (see X-axis of FIG. 13). The Y-axis of FIG. 13 represents cells with the SSTR subtype of antibody treatment. In FIG. 13, the white bar represents antibody (ab) treated with antibody alone; the bar with upward diagonal lines represents treatment with both antibody (ab) and fsk; and the bar with downward diagonal lines represents treatment with ab, fsk and octreotide (Oct). In each treatment, where relevant, 51g/ml anti-hSSTR antibody was used and rabbit IgG was used to bring the final IgG to 10 μg/ml in concentration, 0.5 μM of fsk, and 100 nM Oct. The negative control experiment (Isotype) contains 10 μg/ml of rabbit IgG. In experiments where only 5 μg/ml of anti-hSSTR antibody of the invention was used (e.g., anti-hSSTR antibody subtype 2 or anti-hSSTR antibody subtype 5 only treated experiments), an additional 5 μg/ml of rabbit IgG was added in order to bring the final concentration of IgG to 10 μg/ml.

Table 2 shows the quantification of the data obtained in FIG. 13, summarizing the results in pmol of cAMP per one hundred thousand cells. The anti-hSSTR subtype 2 and anti-hSSTR subtype 5 antibodies did not appear to work like agonists, and instead appeared to possess some antagonist effect in increasing cAMP output. After the cells were treated with forskolin to stimulate cAMP, there was no reduction in cAMP output when the 5 μg/ml anti-hSSTR subtype 2 antibody, the 5 μg/ml anti-hSSTR subtype 5 antibody, and 100 nM octreotide treatments were combined.

BON cells were split and plated in 24-well plates (Costar, Cat#3527) in cell growth media with 10% FBS. Cells were then allowed to grow to 80% in confluence, at which time the media was removed by aspiration, the cells were washed with D-PBS, and BON cell growth media without FBS was added. The cells were allowed to grow in the cell incubator overnight (37° C. with 95% air and 5% CO₂). The next day, cell growth serum without FBS containing 1 mM 3-Isobutyl-1-Methylxanthine (IBMX; Sigma-Aldrich, Cat#15879), a phosphodiesterase inhibitor, was added to prevent degradation of cAMP. Next, treatments, as described above for the cAMP experiment, were applied and allowed to incubate for 5 minutes. At the end of incubation, 0.5 μM of forskolin (Sigma-Aldrich, Cat#6886) was added to all samples to stimulate cAMP production by adenylyl cyclase for an additional 5 minutes in the incubator. Immediately thereafter, the cAMP level was determined by harvesting all samples used in cAMP assay and all media was replaced with 60 ml of 0.1M HCL in order to quench the adenylyl cyclase activity. Next, samples were pipetted up and down to lyses cells and were prepared for cAMP detection. Lysed samples were transferred to 96-well round bottom plates (BD Biosciences, Cat#353918) and spun at 2800 rpm using a Beckman table top centrifuge for ten minutes. Next, supernatants were collected and assayed for cAMP (Cyclic AMP EIA Kit-Caymen Chemicals). For detection, Victor 3V multi-scan reader (Perkin Elmer) was used for absorbance detection set at wavelength 490 nm.

TABLE 2
Data showing adenylyl cyclase activity after treatment
with anti-hSSTR subtype 2 and anti-hSSTR subtype 5
antibodies (see FIG. 13)

| | Growth % | | | |
|---|---|---|---|---|
| | | Anti- | Anti- | |
| | Anti-hSSTR | hSSTR | hSSTR | |
| | subtype 2 and | subtype 5 | subtype 2 | |
| | 5 antibodies | antibodies | antibodies | Isotype |
| Antibody (ab) | 15.4% | 9.9% | 11% | 3.8% |
| Antibody/forskolin | 20.1% | 20.7% | 25.5% | 12.25 |
| (ab/fsk) | | | | |
| Antibody/forskolin/ | 2.03% | 15.1% | 18.1% | 4.8% |
| octreotide (ab/fsk/oct) | | | | |

### Example 6

### The Effect of Anti-hSSTR Antibodies on Other Cell Models

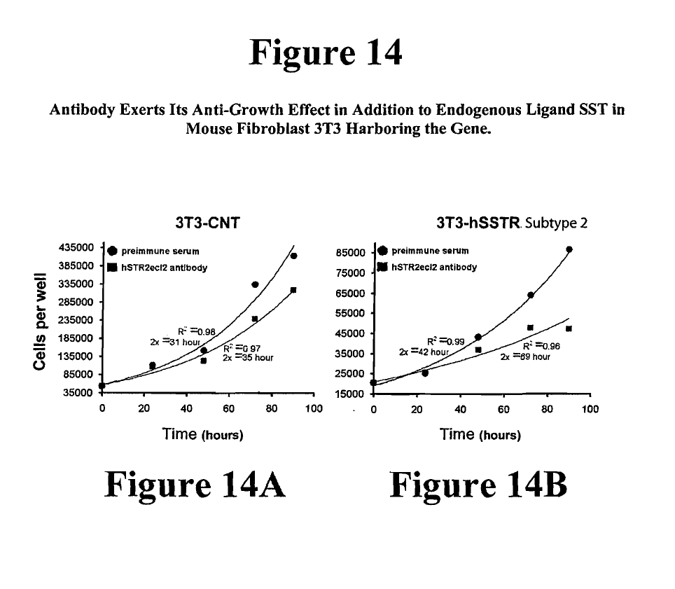

An artificial system using 3T3 Flp-In™ mouse fibroblast cells (Invitrogen) was stably transfected with a SSTR recombinant gene (hSSTR subtype 1, 2, 3, 4 or 5) created to study the somatostatin antibody. These stably transfected 3T3 Flp-In cell lines have been validated to express each SSTR (data not shown). Western blot analysis showed that the anti-hSSTR subtype 2 antibodies only detected hSSTR subtype 2 in the 3T3 Flp-In™ hSSTR subtype 2 cell lysate. The expression of hSSTR subtype 2 protein in 3T3 Flp-In™ hSSTR subtype 2 elicited an intact negative autocrine loop by inducing endogenous mouse SST. The presence of mouse SST imposed strong growth suppression by activating 3T3-Flp-In™ hSSTR subtype 2 (growth suppression is reversible by adding neutralizing mouse anti-SST antibody; data not shown). This growth suppression is shown in FIG. 14. When comparing the doubling time between 3T3-Control (CNT in FIG. 14A; 31 hours) and 3T3 Flp-In™ hSSTR subtype 2 (3T3-hSSTR2 in FIG. 14B; 42 hours), a significant increase was recorded in 3T3 Flp-In™ cells that harbor hSSTR subtype 2. With anti-hSSTR subtype 2 antibody treatment, 3T3-hSSTR subtype 2 increased the doubling time (FIG. 14B) from 35 hours in 3T3-CNT (FIG. 14A) up to 69 hours measured in 3T3-hSSTR subtype 2 when treated with anti-hSSTR subtype 2 antibodies. This growth suppressive effect was reversed by adding hSSTR subtype 2-ecl2 peptide antigen to absorb and neutralize the effect of the anti-hSSTR subtype 2 antibody (data not shown). CellTiter 96® Aqueous One Solution Cell Proliferation Assay (MTS) (Promega, Cat#G3581) was used to measure the growth doubling time for all hSSTR stably transfected Flp-In 3T3 cells (Cat# R761-07) (3T3-CNT and 3T3-hSSTR2) in vitro. The Flp-In 3T3 cells were grown and maintained in D-MEM (Invitrogen, Cat#10567-014) with 10% certified fetal bovine serum (FBS) (Invitrogen, Cat#16000-044) (cell growth media). Flp-In 3T3 cells were first seeded and grown in 96-well culture plates (Costar, Cat#3590) at the density of 3000 cells/well in cell growth media and allowed to attach and grow in 100 μl cell growth media. 24 hours later, cells were treated (either with 10 μg/ml of pre-immune serum or 10 μg/ml of hSSTR2 anti-serum) and measured using MTS assay once per day for the next 96 hours. Daily data collected was used to calculate the doubling time in hours.

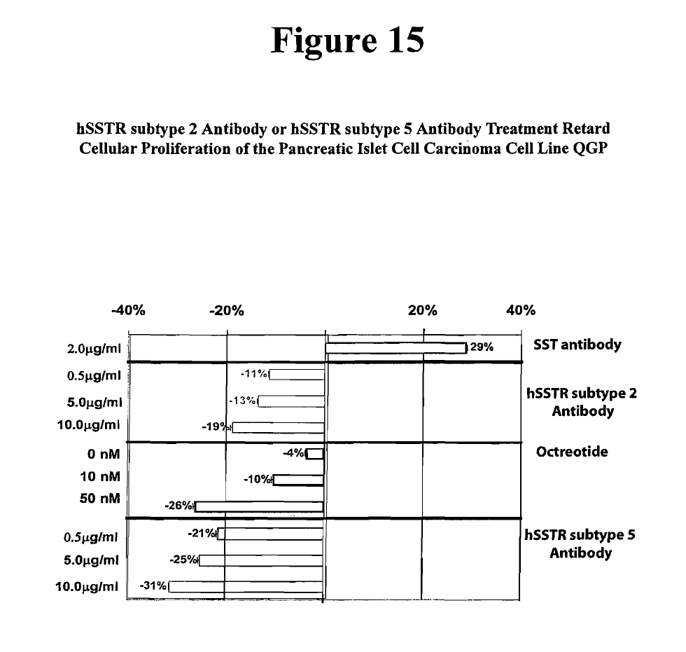

The anti-hSSTR subtype 2 and anti-hSSTR subtype 5 antibodies were examined for their growth inhibition capability in another endogenous SSTR expressing cell line QGP, which is a pancreatic islet carcinoma cell line derived from a non-functional neuroendocrine pancreatic tumor See, Detjen K M, et al. Gastroenterology 2000; 118:735-48), shown in FIG. 15 to possess a negative growth SST-SSTR autocrine loop. The QGP cells were maintained in F12 HAM (Invitrogen, Cat#31765035)/D-MEM (Invitrogen, Cat#10567-014) (50/50) in 10% certified fetal bovine serum (FBS) (Invitrogen, Cat#16000-044) in 10-cm tissue culture plates (BD Biosciences, Cat#353003). QGP cells were first seeded at a density of 5000 cells/well in QGP cell growth media and grown in 96-well cell culture plates (Costar, Cat#3590) and allowed to attach and grow in 100 μl cell growth media. 24 hours later, cells were treated (once every 24 hours with 10 μg/ml of pre-immune serum, 10 μg/ml of anti-hSSTR2 antiserum, 100 nM octreotide or 10 μg/ml of anti-hSSTR5 antiserum) and measured using MTS assay once every 24 hours for the next 96 hours. QGP cells constitutively make their own somatostatin. This negative growth autocrine loop was tested by adding 10 μg/ml neutralizing anti-SST antibody (SST antibody, right hand Y-axis of FIG. 15) once every 24 hours, and a significant growth rescue (29%) was observed. Unlike 3T3-Flp-In SSTR2, QGP is octreotide (100 nM every 24 hour) sensitive even though it makes its own SST. Anti-hSSTR subtype 2 antibodies (5 μg/ml every 24 hours) and anti-hSSTR subtype 5 antibodies (5 μg/ml every 24 hours) caused QGP growth suppression in a dose-responsive fashion. These results further substantiate the conclusion that both anti-hSSTR subtype 2 antibodies and anti-hSSTR subtype 5 antibodies are growth suppressors. CellTiter 96® Aqueous One Solution Cell Proliferation Assay (MTS) (Promega, Cat#G3581), was used as described above to measure the growth doubling time for all QGP cells. Daily data collected was used to calculate the doubling time in hours. QGP cell growth increase or decrease as a result of each treatment is presented as a percent change along the x-axis in FIG. 15. The percent change for QGP cells in each treatment was derived by normalizing treated cells against untreated cells (as % of change in growth) in growth doubling time.

### Example 7

### Inhibition of Serotonin Release in BON Cells by Anti-hSSTR2, 3, and 4 Antibodies

A serotonin (5-Hydroxytryptophan) assay was performed using Serotonin EIA (Biosource, Cat# KAPL10-0900) to measure serotonin release by BON cells over a 48-hour period with or without treatment. Initially, 2 million BON cells were seeded in 10-cm tissue culture plates (BD Biosciences, Cat#353003) and grown for 24 hours in 10 ml of F12 HAM (Invitrogen, Cat#31765035)/D-MEM (Invitrogen, Cat#10567-014) (50/50) in 10% certified fetal bovine serum (FBS) (Invitrogen, Cat# 16000-044) (cell growth media). After 24 hours, the BON cells were subjected to six different daily treatments (at maximum dosages) and grown in FBS-free cell growth media for 2 days in a sterile tissue culture chamber at 37° C. with 6% CO₂. These six treatments were: (1) Vehicle (PBS), (2) 100 nM octreotide (Oct) (Sigma-Aldrich, Cat# 01014), (3) 5 μg/ml rabbit IgG (Vector labs, Cat# 11000), (4) 5 μg/ml anti-hSSTR2 antibody, (5) 5 μg/ml anti-hSSTR3 antibody, and (6) 5 μg/ml anti-hSSTR2 antibody. The anti-hSSTR Abs (4)-(6) were antibodies produced in the experiment described in Example 1. At the end of each treatment, 1× Halt Protease Inhibitor cocktail (Pierce, Cat# 78415) was immediately added to each sample and the media was collected from each sample and used for Serotonin detection.

Next, cells were dissociated from the plate using 2 ml of 1× trypsin-EDTA (Invitrogen Cat#2520) in cell growth media and incubated at 37° C. with 6% CO₂ for 5 minutes. Subsequently, an additional 8 ml of cell growth media was added to neutralize trypsin-EDTA, and subjected to 1000 rpm at 4° C. in 15 ml Falcon tubes using Beckman table top centrifuge. Guava Technologies PCA-96 instrument (tabletop flow cytometer) and Viacount flex reagent (Guava Technologies, Cat# 4500-0100) were then used to determine the number of cells for the abovementioned treatments. The results were as follows: (1) 8 million cells for PBS treated samples, (2) 7 million cells for 100 nM octreotide treated samples, (3) 7.9 million cells for 5 μg/ml rabbit IgG treated samples, (4) 4.9 million cells for 5 μg/ml anti-hSSTR2 antibody treated samples, (5) 5.8 million cells for 5 μg/ml anti-hSSTR3 antibody treated samples, and (6) 5.8 million cells for 5 μg/ml anti-hSSTR5 antibody treated samples.

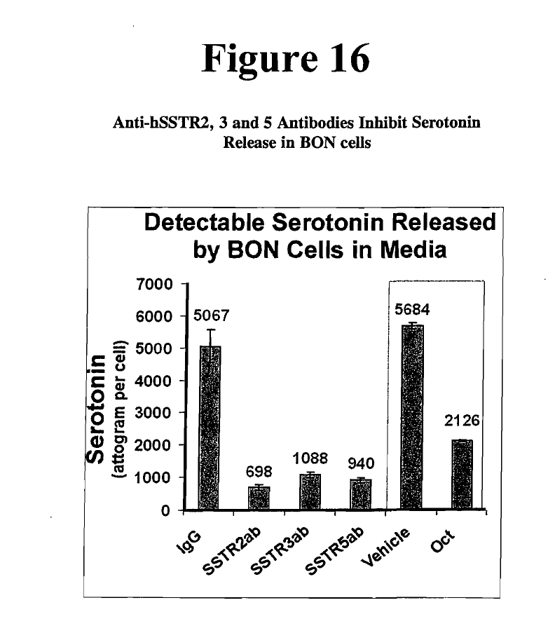

The cell numbers obtained for each treatment were used to calculate the release of serotonin per cell in attograms over a 48-hour period. The results shown in FIG. 16 represent four independent experiments and the standard deviation was plotted as a vertical line with top and bottom limits as short horizontal lines on top of each bar of the bar chart. The serotonin level in the media for rabbit IgG treated samples (5056 attogram/cell) compared to the anti-hSSTR antibodies treated samples showed: (1) a decrease of 7.2 fold of serotonin in BON cells when treated with anti-hSSTR2 antibody (698 attogram/cell), (2) a decrease of 4.7 fold of serotonin level when treated with anti-hSSTR3 antibody in the media (1088 attogram/cell), and (3) a decrease of 5.4 fold of serotonin release when treated with anti-hSSTR5 antibody in the media (940 attogram/cell). Interestingly, the serotonin level in the octreotide treated samples' media (2126 attogram/cell) when compared to its respective control sample (vehicle, PBS treated) (5684 attogram/cell) yielded a 2.7 fold of decrease in serotonin release.

In summary, although each anti-hSSTR antibody demonstrated different potencies in inhibiting serotonin release, all three antibodies demonstrated a significant ability to inhibit serotonin release. Additionally, all antibody treatments demonstrated a superior ability to inhibit serotonin release relative to octreotide at maximum dosage.

While the present invention has been described with reference to the specific embodiments thereof, it should be understood by those skilled in the art that various changes may be made, and equivalents may be substituted, without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

What is claimed is:

1. An isolated polyclonal antibody or antigen-binding portion thereof that specifically binds to amino acid residues 1-14 of SEQ ID NO: 1 in the extracellular loop 2 of a human somatostatin receptor (hSSTR) subtype 2.

2. The antibody or antigen-binding portion of claim 1, which has agonist-like or antagonist-like properties on the hSSTR subtype 2.

3. The antibody or antigen-binding portion of claim 1, which has agonist-like properties on the hSSTR subtype 2.

4. The antibody or antigen-binding portion of claim 1, which possesses both agonist-like and antagonist-like properties on the hSSTR subtype 2.

5. An isolated polyclonal antibody or antigen-binding portion thereof that specifically binds to amino acid residues 1-14 of SEQ ID NO: 1 in the extracellular loop 2 of hSSTR, wherein the antibody or antigen binding portion binds to an epitope within the amino acid sequence of SEQ ID NO: 1 in the extracellular loop 2 of the hSSTR subtype 2.

6. The antibody or antigen-binding portion of claim 1, which, upon contact with a cell or a biological sample expressing a human SSTR, results in at least one effect on a cell or the tissue of the biological sample compared to a cell or a tissue expressing a human SSTR that has not been contacted with the antibody or the antigen-binding portion, which effect is selected from the group consisting of:
a. suppression of serotonin release;
b. induction of activation of caspase 8;
c. induction of activation of caspase 9;
d. suppression of cell growth;
e. increase in cell death;
f. increase in programmed cell death by apoptosis;
g. induction of cell cycle arrest of the cell by maintaining the cell in the G0/G1 phase;
h. induction of cell cycle arrest of the cell by maintaining the cell in the G2/M phase;
i. induction of cell cycle arrest of the cell by preventing the cell from entering the S phase; and
j. suppression of neuroendocrine dense core granular release.

7. The antibody or antigen-binding portion of claim 1, wherein upon contact with the antibody or antigen-binding portion, a cell expressing a human SSTR produces a greater level of cAMP than is produced by said cell expressing a human SSTR that has instead been contacted with octreotide.

8. The antibody or antigen-binding portion of claim 6, which results in at least two effects selected from the group consisting of (a)-(i).

9. The antibody or antigen-binding portion of claim 6, which results in at least three effects selected from the group consisting of (a)-(i).

10. The antibody or antigen-binding portion of claim 1, which is detectably labeled.

11. The labeled antibody or antigen-binding portion of claim 10, wherein the detectable label is selected from the group consisting of: (a) an enzyme; (b) a radioisotope; (c) a fluorescent label; and (d) a paramagnetic moiety.

12. The antibody or antigen-binding portion of claim 1, which is conjugated to a chemotherapeutic agent.

13. A method for producing a polyclonal antibody or antigen-binding portion thereof that specifically binds to amino acid residues 1-14 of SEQ ID NO: 1 in the extracellular loop 2 of hSSTR subtype 2, which comprises:
a. immunizing a mammal with a polypeptide comprising amino acid residues 1-14 of SEQ ID NO: 1; and
b. isolating an antibody or an antigen-binding portion thereof that specifically binds to amino acid residues 1-14 of SEQ ID NO: 1 in the extracellular loop 2 of the hSSTR subtype 2.

14. A method of suppressing serotonin release in a cell, which comprises contacting a cell expressing a human SSTR with an effective amount of an antibody or antigen-binding portion of claim 1 to suppress serotonin release.

15. A method of inducing cell cycle arrest, which comprises contacting a cell expressing a human SSTR with an effective amount of an antibody or antigen-binding portion of claim 1 that induces cell cycle arrest.

16. The method of claim 14 or 15, wherein the antibody or antigen-binding portion is detectably labeled.

17. A method for detecting the presence of a human SSTR subtype 2 comprising amino acids 1-14 of SEQ ID NO: 1 in a biological sample which comprises:
a. contacting the biological sample with an antibody or antigen-binding portion of claim 1 under conditions which permit the specific binding of the antibody or antigen-binding portion to the SSTR subtype 2 to form a complex, and
b. detecting the presence of the complex, wherein the presence of the complex indicates the presence of a human SSTR subtype 2.

18. A pharmaceutical composition comprising the antibody or antigen-binding portion of claim 1 and a pharmaceutically acceptable carrier.

19. A method of treating cancer in a patient, which comprises: (a) identifying a patient having cancer cells expressing a human SSTR subtype 2; and (b) administering to the patient a therapeutically effective amount of the antibody or antigen-binding portion according to claim 1.

20. The method of claim 19, wherein the cancer is a neuroendocrine tumor.

21. The method of claim 20, wherein the neuroendocrine tumor is selected from the group consisting of: adrenal pheochromocytoma, gastrinoma, glucagonoma, insulinoma, medullary carcinoma of the thyroid, multiple endocrine neoplasia syndrome, pancreatic endocrine tumors, paragangliomas, vasoactive intestinal polypeptide tumors, calcitoninoma, neurotensinoma, parathyroid hormone-related peptide tumor, Merkel cell cancer, small-cell lung cancer, neuroblastoma, and Carney's complex.

22. The method of claim 19, wherein the antibody or antigen-binding portion is conjugated to a chemotherapeutic agent.

23. The method of claim 22, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

24. The method of claim 19, further comprising administering to the patient a second therapeutic agent to treat the cancer.

25. The method of claim 24, wherein the second therapeutic agent is radiation or a chemotherapeutic agent.

26. The method of claim 25, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

27. A method of treating carcinoid syndrome in a patient, comprising the steps of:
a. identifying a patient as having carcinoid syndrome; and
b. administering to the patient a therapeutically effective amount of an antibody or antigen-binding portion according to claim 1.

28. The method of claim 27, wherein the antibody or antigen-binding portion is conjugated to a chemotherapeutic agent.

29. The method of claim 28, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

30. The method of claim 28, further comprising administering to the patient a second therapeutic agent to treat carcinoid syndrome.

31. The method of claim 30, wherein the second therapeutic agent is selected from the group consisting of radiation, a somatostatin analogue, and a chemotherapeutic agent.

32. The method of claim 31, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

33. An isolated polyclonal antibody or antigen-binding portion thereof that specifically binds to amino acid residues 1-14 of SEQ ID NO: 2 in the extracellular loop 2 of a hSSTR subtype 5.

34. The antibody or antigen-binding portion of claim 33, which has agonist-like or antagonist-like properties on the hSSTR subtype 5.

35. The antibody or antigen-binding portion of claim 33, which has agonist-like properties on the hSSTR subtype 5.

36. The antibody or antigen-binding portion of claim 33, which possesses both agonist-like and antagonist-like properties on the hSSTR subtype 5.

37. An isolated polyclonal antibody or antigen-binding portion that specifically binds to amino acid residues 1-14 of SEQ ID NO: 2 in the extracellular loop of a hSSTR subtype 5, wherein the antibody or antigen-binding portion binds to an epitope within the amino acid sequence of SEQ ID NO: 2 in the extracellular loop 2 of the hSSTR subtype 5.

38. The antibody or antigen-binding portion of claim 33, which, upon contact with a biological sample expressing a human SSTR, results in at least one effect on a cell or a tissue of the biological sample compared to a cell or a tissue expressing a human SSTR that has not been contacted with the antibody or antigen-binding portion, which effect is selected from the group consisting of:
a. suppression of serotonin release;
b. induction of activation of caspase 8;
c. induction of activation of caspase 9;
d. suppression of cell growth;
e. increase in cell death;
f. increase in programmed cell death by apoptosis;
g. induction of cell cycle arrest of the cell by maintaining the cell in the G0/G1 phase;
h. induction of cell cycle arrest of the cell by maintaining the cell in the G2/M phase;
i. induction of cell cycle arrest of the cell by preventing the cell from entering the S phase; and
j. suppression of neuroendocrine dense core granular release.

39. The antibody or antigen-binding portion of claim 33, wherein upon contact with the antibody or antigen-binding portion, a cell expressing a human SSTR produces a greater level of cAMP than is produced by said cell expressing a human SSTR that has instead been contacted with octreotide.

40. The antibody or antigen-binding portion of claim 38, which results in at least two effects selected from the group consisting of (a)-(i).

41. The antibody or antigen-binding portion of claim 38, which results in at least three effects selected from the group consisting of (a)-(i).

42. The antibody or antigen-binding portion of claim 33, which is detectably labeled.

43. The labeled antibody or antigen-binding portion of claim 42, wherein the detectable label is selected from the group consisting of: (a) an enzyme; (b) a radioisotope; (c) a fluorescent label; and (d) a paramagnetic moiety.

44. The antibody or antigen-binding portion of claim 33, which is conjugated to a chemotherapeutic agent.

45. A method for producing a polyclonal antibody or antigen-binding portion thereof that specifically binds to amino acid residues 1-14 of SEQ ID NO: 2 in the extracellular loop 2 of hSSTR subtype 5, which comprises:
a. immunizing a mammal with a polypeptide comprising amino acid residues 1-14 of SEQ ID NO: 2; and
b. isolating an antibody or an antigen-binding portion thereof that specifically binds to amino acid residues 1-14 of SEQ ID NO: 2 in the extracellular loop 2 of the hSSTR subtype 5.

46. A method of suppressing serotonin release in a cell, which comprises contacting a cell expressing a human SSTR with an effective amount of an antibody or antigen-binding portion of claim 33 that suppresses serotonin release.

47. A method of inducing cell cycle arrest in a cell, which comprises contacting a cell expressing a human SSTR with an effective amount of an antibody or antigen-binding portion of claim 33 that induces cell cycle arrest.

48. The method of claim 46 or 47, wherein the antibody or antigen-binding portion is detectably labeled.

49. A method for detecting the presence of a human SSTR subtype 5 comprising amino acids 1-14 of SEQ ID NO: 2 in a biological sample which comprises:
a. contacting the biological sample with an antibody or antigen-binding portion of claim 1 under conditions which permit the specific binding of the antibody or antigen-binding portion to the SSTR subtype 5 to form a complex, and
b. detecting the presence of the complex, wherein the presence of the complex indicates the presence of a human SSTR subtype 5.

50. A pharmaceutical composition comprising the antibody or antigen-binding portion of claim 33 and a pharmaceutically acceptable carrier.

51. A method of treating cancer in a patient, which comprises:
a. identifying a patient as having cancer cells expressing a human SSTR subtype 5; and
b. administering to the patient a therapeutically effective amount of the antibody or antigen-binding portion according to claim 33.

52. The method of claim 51, wherein the cancer is a neuroendocrine tumor.

53. The method of claim 52, wherein the neuroendocrine tumor is selected from the group consisting of: adrenal pheochromocytoma, gastrinoma, glucagonoma, insulinoma, medullary carcinoma of the thyroid, multiple endocrine neoplasia syndrome, pancreatic endocrine tumors, paragangliomas, vasoactive intestinal polypeptide tumors, calcitoninoma, neurotensinoma, parathyroid hormone-related peptide tumor, Merkel cell cancer, small-cell lung cancer, neuroblastoma, and Camey's complex.

54. The method of claim 51, wherein the antibody or antigen-binding portion is conjugated to a chemotherapeutic agent.

55. The method of claim 54, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

56. The method of claim 51, further comprising administering to the patient a second therapeutic agent to treat the cancer.

57. The method of claim 56, wherein the second therapeutic agent is radiation or a chemotherapeutic agent.

58. The method of claim 57, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

59. A method of treating carcinoid syndrome in a patient, comprising the steps of:
a. identifying a patient as having carcinoid syndrome; and
b. administering to the patient a therapeutically effective amount of an antibody or antigen-binding portion according to claim 33.

60. The method of claim 59, wherein the antibody or antigen-binding portion is conjugated to a chemotherapeutic agent.

61. The method of claim 60, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.

62. The method of claim 60, further comprising administering to the patient a second therapeutic agent to treat carcinoid syndrome.

63. The method of claim 61, wherein the second therapeutic agent is selected from the group consisting of radiation, a somatostatin analogue, and a chemotherapeutic agent.

64. The method of claim 63, wherein the chemotherapeutic agent is selected from the group consisting of an alkylating agent, an anti-metabolite, an antibiotic, a plant-derived anti-tumor agent, a platinum-coordinated compound, a tyrosine kinase inhibitor, a growth factor inhibitor, an anti-angiogenesis agent, a mitotic inhibitor, a cell cycle inhibitor, a topoisomerase inhibitor, and an interferon.


SEQUENCE LISTING (10 sequences)

SEQ ID NO: 1 - protein, 14 aa, Homo sapiens
QWGRSSCTINWPGE

SEQ ID NO: 2 - protein, 14 aa, Homo sapiens
DVQEGGTCNASWPE

SEQ ID NO: 3 - protein, 14 aa, Homo sapiens
TVACNMLMPEPAQR

SEQ ID NO: 4 - protein, 14 aa, Homo sapiens
PRGMSTCHMQWPEP

SEQ ID NO: 5 - protein, 14 aa, Homo sapiens
DTRPARGGQAVACN

SEQ ID NO: 6 - protein, 28 aa, Homo sapiens
AGLRSNQWGRSSCTINWPGESGAWYTGF

SEQ ID NO: 7 - protein, 21 aa, Homo sapiens
ADVQEGGTCNASWPEPVGLWG

SEQ ID NO: 8 - protein, 25 aa, Homo sapiens
SRTAANSDGTVACNMLMPEPAQRWL

SEQ ID NO: 9 - protein, 26 aa, Homo sapiens
FSGVPRGMSTCHMQWPEPAAAWRAGF

SEQ ID NO: 10 - protein, 25 aa, Homo sapiens
ADTRPARGGQAVACNLQWPHPAWSA